(19) United States
(12) Patent Application Publication
(10) Pub. No.: US 2023/0045590 A1
(43) Pub. Date: Feb. 9, 2023

(54) GENETICALLY CORRECTED CELLS FOR THERAPEUTIC USE

(71) Applicant: The Board of Trustees of the Leland Stanford Junior University, Stanford, CA (US)

(72) Inventors: Anthony E. Oro, Stanford, CA (US); Marius Wernig, Palo Alto, CA (US)

(21) Appl. No.: 17/786,359

(22) PCT Filed: Dec. 21, 2020
(86) PCT No.: PCT/US2020/066388

§ 371 Date: Jun. 16, 2022

Related U.S. Application Data
(60) Provisional application No. 62/952,869, filed on Dec. 23, 2019.

(51) Int. Cl. C12N 5/071; C12N 5/074; C12N 9/22; C12N 15/11; C12N 15/90; A61K 35/36; A61P 17/00

(52) U.S. Cl. CPC C12N 5/063; C12N 5/0696; C12N 9/22; C12N 15/11; C12N 15/907; A61K 35/36; A61P 17/00; C12N 2501/602; C12N 2501/604; C12N 2501/606; C12N 2506/1307; C12N 2506/45; C12N 2501/608; C12N 2310/20

(57) ABSTRACT

Compositions and methods are provided for production of cells useful in regenerative therapies.

### CROSS REFERENCE

This application claims priority to U.S. Provisional Application No. 62/952,869, filed Dec. 23, 2019, which is incorporated herein in its entirety for all purpose.

### BACKGROUND

Pluripotential stem cells can self-renew and differentiate into somatic tissues, providing a source of cells for regenerative therapies. Induced pluripotent stem cells (iPSC) in particular can be generated from somatic tissues, thereby providing a supply of patient specific cells. As genetic manipulation of pluripotent cells and the subsequent transplantation allow genetic correction and selection, these techniques hold great promise in producing gene-modified cells and in treating genetic diseases.

The CRISPR-Cas9 system has enabled rapid genome editing in different species at a very high efficiency and specificity. CRISPR-Cas9-mediated genome editing requires only a short single-guide RNA (sgRNA) to guide site-specific DNA recognition and cleavage, resulting in gene modification at a target locus via nonhomologous end joining (NHEJ)-mediated insertions/deletions (indels) or homology-directed repair (HDR) based on an exogenously supplied oligonucleotide. This system is relatively easy to implement compared to other genetic editing techniques, and may provide opportunities for clinically relevant methods.

Epidermolysis bullosa (EB) represents a group of debilitating inherited skin disorders in which blisters develop after relatively minor trauma to the skin. Symptoms and severity depend on the type of EB. Recessive dystrophic epidermolysis bullosa (RDEB), with an estimated incidence of 1 per million live births in the United States, is the most severe form of EB caused by absence of a protein known as type VII collagen. The COL7A1 locus encodes type VII collagen, the main component of the anchoring fibrils which tether the epidermis to the dermal tissue underneath. Patients with RDEB develop large, severely painful blisters and open wounds, leaving affected children with painful chronic wounds over their body, and risk of death from cancer later in life. The extensive blistering and open wounds on the skin greatly affect patient quality of life, with patients reporting severe daily pain, especially during wound care.

While loss-of-function mutations in the COL7A1 locus occur throughout the gene, there are several COL7A1 alleles in founder populations in North and Central America that exist within large groups of patients within the EB Registry. This includes three alleles emanating from Central America: the Colorado mutation (7485+5G>A), deriving from a central American and European origin, and two alleles originating in distinct Mexican populations now residing in the United States. The availability of RDEB patients with such genotypes make these alleles ideal targets for novel targeted cell and gene therapy approaches.

There are currently no therapies approved for the treatment of RDEB. Treatments are only supportive in nature and include prevention of trauma to the skin, promoting good wound care, treatment of skin infections, promotion and support of nutrition, treatment of anemia and skin cancer surveillance. The basic underlying tenets of care for all EB patients are avoidance of blistering (by meticulous protective padding/dressings used on the skin), prevention of secondary infection, and promotion of an occlusive wound healing environment through the daily application of various types of non-adhesive dressings to large areas of the body. Wound supplies can cost over ten thousand dollars per month. However, despite these careful measures, substantial areas of denuded skin foster bacterial growth and can lead to infection. In addition, chronic wounds and erosions contribute to anemia, pain, itch, and failure to thrive. Previous epidemiological studies indicate that patients with the severe generalized variant of RDEB that survive into late adulthood are at heightened risk for invasive squamous cell carcinoma (SCC), with 55% dying from SCC by age 40. Since the epidermis is continuously renewed by stem cells in the proliferative basal layer, a permanent corrective therapy for EB must correct the COL7A1 gene in this stem cell population.

Methods for producing genetically corrected pluripotent stem cells, and the use of such cells in treating genetic diseases such as EB, are of great interest.

### SUMMARY

Methods are provided for integration-free, xeno-free, feeder-free single clonal step production of CRISPR-corrected iPSC cells from fibroblasts. In some preferred embodiments the cells are autologous with respect to an individual selected for treatment. In other embodiments the cells are allogeneic with respect to an individual selected for treatment. The cells thus obtained may meet the requirements for clinical use, i.e. the cells are cGMP compatible. Engineering iPSC allows patient samples to be obtained from cells such as fibroblasts, which may be more available than cells from affected tissues. Further, the iPSC samples can be stored frozen for long periods of time, and expanded in suitable numbers for multiple administrations of a tissue graft.

In some embodiments a composition of genetically corrected integration-free, cGMP compatible iPSC is provided. The cells may be genetically corrected, for example, at a wide range of loci involved in the cause of disease, e.g. in muscle diseases, hematopoietic diseases, metabolic diseases, skin diseases, etc. Specific loci include, without limitation, dystrophin mutations involved in muscular dystrophy, for example at exon 51; hemoglobins involved in thalassemia or sickle cell anemia, for example beta-globin; targeting CCR5 for the treatment of HIV infection; Swedish APP5 in early onset Alzheimers disease; huntingtin mutations associated with Huntington's disease; mutations associated with retinitis pigmentosa; LCA10 mutations associated with Leber congenital amaurosis; etc.

The manufacture of the genetically corrected iPSCs is optimized through the development of an integration-free, feeder-free, xeno-free, single clonal step method using preformed ribonuclear protein (RNPs) complexes with high-fidelity CAS9 and guide RNAs and mRNAs encoding reprogramming factors. The combination of footprint-free reprogramming and gene editing of patient somatic cells into a one-step process is dependent on the ability to efficiently generate iPSCs using non-integration-based methods and the development of high-efficiency gene editing.

In some embodiments the reprogramming to pluripotency utilizes synthetic capped mRNAs containing modified nucleobases (modified mRNA). In some embodiments the reprogramming factors comprise a modified version of Oct4 fused with the MyoD transactivation domain (called M3O), Sox2, Klf4, cMyc, and Lin28A. In some embodiment the reprogramming is performed with a feeder-free system in xeno-free media. The mRNA reprogramming method provides for robust iPS cell colony formation in patient fibroblast lines.

The reprogramming step above is combined with genetic correction in a single step. Genetic correction is performed with CRISPR-associated CAS9 nucleases, to induce a precise DNA double-strand break (DSB) at endogenous pathogenic genomic loci with high efficiency. The advantages of this approach include the lack of integrating viruses or clonal selection, and the ability to perform both reprogramming and mutational correction in one step. The CAS9 protein is delivered by mRNA transfection (mRNA) with RNP complexes, and may utilize high-fidelity CAS9 protein in RNP complexes. The fibroblasts are transfected with RNPs and DNA oligonucleotides, e.g. from about 5 to about 25 pmole of guide mRNA and CAS9 protein. After from about 2 to about 6, about 3 to about 5, about 4 days in culture, cells are reprogrammed by repeated transfections with the mRNAs encoding the reprogramming factors in defined reprogramming media. iPS cell colonies are screened for genetic correction, e.g. by droplet digital PCR, direct sequencings, etc. Cells identified as having the correct genotype are expanded as iPSC and may be frozen for future use, or differentiated into somatic cells.

In some embodiments the cells are obtained from an individual with recessive dystrophic epidermolysis bullosa (RDEB), and a defect at the COL7A1 locus is corrected by an integration-free method to provide a normally functional collagen VII protein.

In some embodiments compositions and methods are provided for the treatment of EB in a human subject. Genetically corrected iPSCs obtained by the methods described herein can be grown in large quantities and be induced to differentiate into genetically corrected iPSC-derived keratinocyte stem cells, herein called iKCs. iKCs resemble and functional like somatic patient keratinocytes, but are corrected at the COL7A1 locus. A further improvement is provided by the selection of graftable iKCs for expansion. Expression of CD49f (ITGA6)^{HI} cells is shown to be an in-process potency marker that correlates with iKCs and tissue graftability. ITGA6 is one of the earliest surface markers to arise in definitive mature keratinocytes and the ITGA6 bright population correlates with a K14⁺, K18⁻ expression basal keratinocyte phenotype. Importantly ITGA6 bright cells displayed enhanced graftability, resulting in an organotypic epidermis that displays proper polarity and differentiation. Selection may utilize one or more of flow cytometry, magnetic bead selection, and the like, with an affinity reagent, e.g. an antibody, specific for human CD49f.

Clinical scaling and manufacturing of patient iKC sheets is achieved by differentiating patient-derived, COL7A1-corrected iPSCs. In some embodiments the level of iKC COL7A1 expression is greater than normal human keratinocyte levels of expression. In some embodiments, the level of iKC COL7A1 expression is smaller, similar, or same as normal human keratinocyte levels of COL7A1 expression. Included in the invention is an isolated population of iKCs engineered and selected by the methods described herein to express wild-type collagen VII, which may be provided in a pharmaceutical unit dose composition. In some embodiments, the subject is a human suffering from a genetic defect in collagen VII causing EB. In the embodiments, the genetic defect is RDEB.

In some embodiments, a graftable epithelial sheet composition is provided. To generate the sheet composition, a defined cell product can be obtained following in vitro expansion of iKC. Optionally the process includes selection for desired cells types, for example by selecting cells with flow cytometry, magnetic bead selection, etc. The composition desirably comprises greater than about 50% iKCs, which can be determined by, for example expression of CD104; and may be greater than 55%, greater than 60%, or more. The composition desirably comprises less than about 35% fibroblast feeder cells, which can be determined by, for example expression of CD90; and may be less than 30%, less than 25%, or less. The composition desirably comprises less than about 1% undifferentiated iPSCs, which can be determined by, for example expression of Tra1-60; and may be less than 0.5%%, less than 0.1%, or less. After expansion, the defined cell composition is placed in culture, grown to confluence and then released and delivered to the operating room.

In some embodiments of the invention, a method is provided for treatment of EB, the method comprising, consisting essentially of, or yet further consisting of, producing a genetically corrected population of iPSC from a subject suffering from EB by the methods described herein, differentiating the iPSC to iKCs and reintroducing the iKC-derived sheets into the individual. In some embodiments the iKCs are selected for expression of CD49f prior to manufacture of keratinocyte sheets for grafting. The genetically corrected iKCs are cultured to generate a sheet of from about 25 cm² to about 100 cm² for grafting. The iKCs sheets are placed on uninfected, eroded, and/or scarred wound sites that lacks clinical evidence of squamous cell carcinoma (SCC). Wound sites may be from about 50 cm², from about 100 cm², and/or from about 200 cm². In some embodiments, wounds are generated for grafts. In some such embodiments, the wound is electrocauterized to ablate residual non-corrected wound bed keratinocytes. Grafts are affixed to wound beds via dissolvable sutures following wound bed preparation.

In another embodiment, the disclosure provides a pharmaceutical composition comprising, consisting essentially of, or yet further consisting of an iKC sheet, which comprises, consists essentially of, or yet further consists of iKCs genetically corrected with an integration-free method. In some embodiments the iKC sheet displays proper polarity and differentiation for epidermal grafts. In some embodiments, the iKC sheet is placed on a bioengineered skin equivalent. In some embodiments, the iKC sheet is placed on an acellular matrix, a collagen matrix, an ECM protein or chemical layer, or a biocompatible mesh. In one embodiment, the acellular matrix is made of human and/or animal dermis. In some embodiments, the biocompatible mesh is made of thermoplastic resin, polyethylene, ultra-high molecular weight polyethylene, high molecular weight polyolefin, uncoated monofilament polypropylene, polyether ether ketone, polyethylene terephthalate, polytetrafluoroethylene, expanded polytetrafluoroethylene, nylon, silicon, or any combination thereof.

### BRIEF DESCRIPTION OF THE DRAWINGS

FIG. 1. Functional human epidermis from FACS sorted ITGA6 bright iKCs in A) in vitro organotypic cultures and (B,C) 4 week in vivo grafts. Grafts stained with K14, K10 loricrin, and ITGA6 (red) and human-specific Collagen VII (LH7.2, green) B) H+E staining of iKC (left) or neonatal keratinocytes (right). C) in vivo grafts.

FIG. 2. Characterization of corrected IPS Intermediates. A) Droplet digital PCR using a Taqman probe specific for the corrected allele (blue) and a Taqman probe specific for a biallelic locus (RPP30; green) demonstrates non-mosaic iPSC intermediates (i.e. iPSC clones R-10 & R-24). B) The COL7A1 locus undergoes PCR and Sanger sequencing and karyotyping. Columns show reprogramming efficiency, number of candidate colonies with less than 5% abnormal clones, and karyotype analysis.

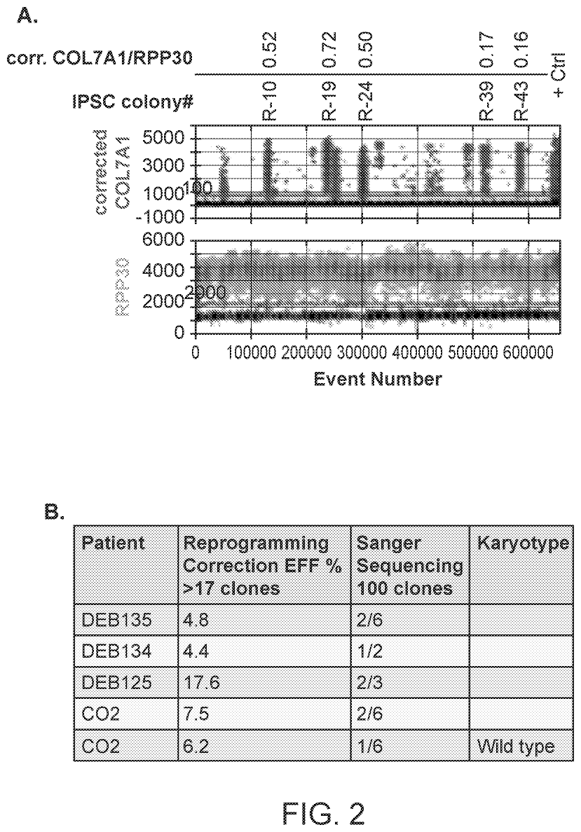

FIG. 3. Reproducible line-to-line differentiation using DEBCT2018 manufacturing. Coupling efficiency (CE) defined by ITGA6 bright cells at Day 45 differentiation/PI#3.

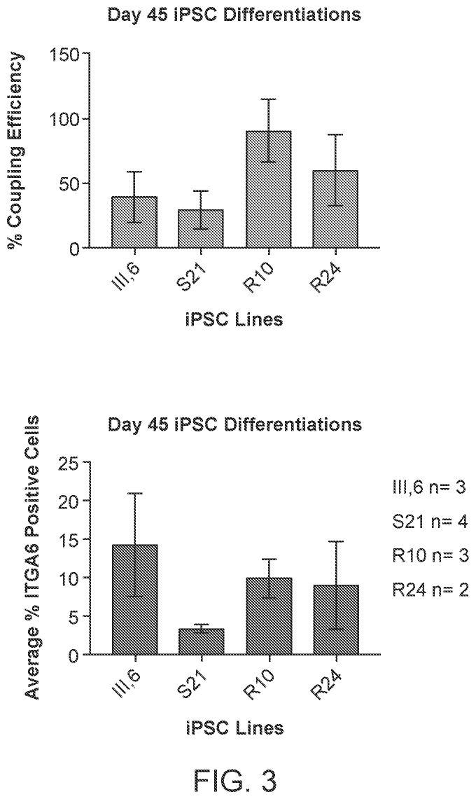

FIG. 4. (A) Sorting ITGA6 bright (red) keratinocyte-like cells at Day 50 result in (B-D) K18 low basal keratinocytes that have polarized epidermal grafts; by contrast (E-G) ITGA6 dull (blue) cells display higher K18 expression, contain mixed cell morphologies, and display poor graftability.

FIG. 5. Selection for CD49f positive cells.

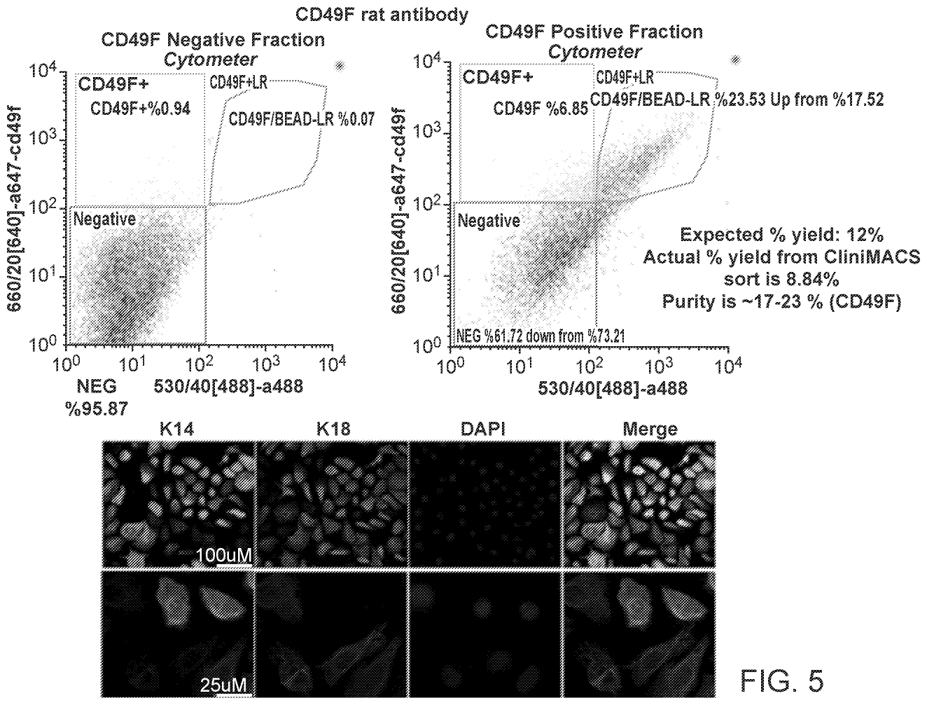

FIG. 6. Workflow for iKC differentiation.

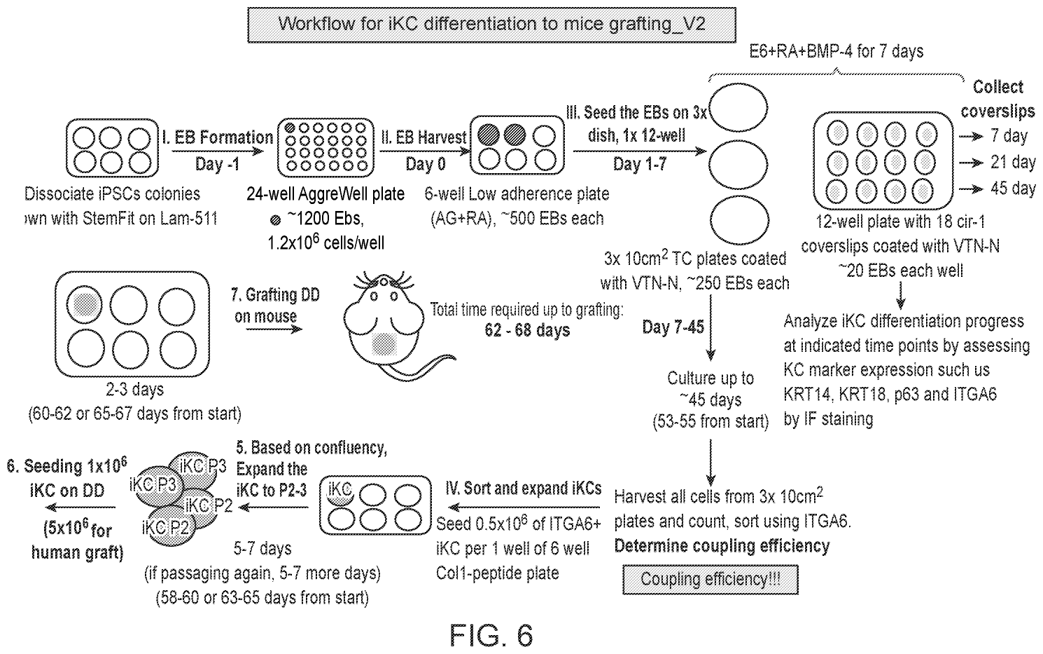

FIG. 7: iKCs after 39 days starting differentiation.

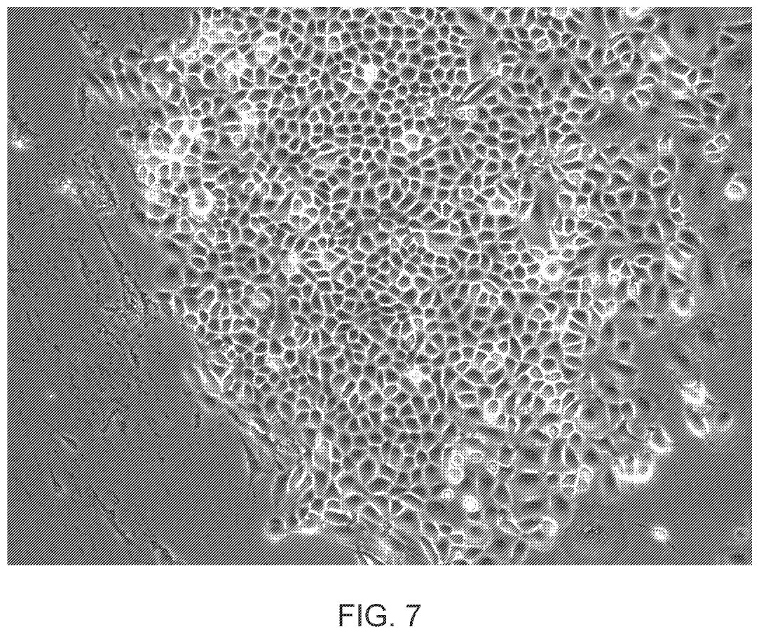

### DETAILED DESCRIPTION

It is to be understood that this invention is not limited to the particular methodology, protocols, cell lines, animal species or genera, and reagents described, as such may vary. It is also to be understood that the terminology used herein is for the purpose of describing particular embodiments only and is not intended to limit the scope of the present invention, which will be limited only by the appended claims.

As used herein, the singular forms “a,” “an,” and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

Any embodiment of any of the present methods, devices, and systems may consist of, or consist essentially of—rather than comprise/include/contain/have—the described steps and/or features. Thus, in any of the claims, the term “consisting of” or “consisting essentially of” may be substituted for any of the open-ended linking verbs recited above, in order to change the scope of a given claim from what it would otherwise be using the open-ended linking verb.

The use of the term “or” in the claims is used to mean “and/or” unless explicitly indicated to refer to alternatives only, or the alternatives are mutually exclusive, although the disclosure supports a definition that refers to only alternatives and “and/or.”

Throughout this application, the term “about” is used to indicate that a value includes the standard deviation of error for the device or method being employed to determine the value.

By “pluripotency” and pluripotent stem cells it is meant that such cells have the ability to differentiate into all types of cells in an organism. The term “induced pluripotent stem cell” encompasses pluripotent cells, that, like embryonic stem (ES) cells, can be cultured over a long period of time while maintaining the ability to differentiate into all types of cells in an organism, but that, unlike ES cells (which are derived from the inner cell mass of blastocysts), are derived from differentiated somatic cells, that is, cells that had a narrower, more defined potential and that in the absence of experimental manipulation could not give rise to all types of cells in the organism. By “having the potential to become iPS cells” it is meant that the differentiated somatic cells can be induced to become, i.e. can be reprogrammed to become, iPS cells. In other words, the somatic cell can be induced to dedifferentiate so as to establish cells having the morphological characteristics, growth ability and pluripotency of pluripotent cells. iPS cells have an hESC-like morphology, growing as flat colonies with large nucleo-cytoplasmic ratios, defined borders and prominent nuclei. In addition, iPS cells express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, the iPS cells are capable of forming teratomas. In addition, they are capable of forming or contributing to ectoderm, mesoderm, or endoderm tissues in a living organism.

Modified mRNA (mmRNA) composition. In the methods of the invention, one form of nuclear reprogramming is accomplished by contacting somatic cells with a cocktail of mmRNA encoding reprogramming factors. The modified mRNA may be provided as a purified IVT transcript. Various combinations and ratios of reprogramming factors are known and used in the art, including, for example, for nuclear reprogramming of a somatic cell to pluripotency, one may use a cocktail comprising the five reprogramming factors (OKSML; Oct 4, KLF4, Sox2, cMyc and Lin28) in equimolar quantities except for Oct 4. Preferred is the use of a fusion gene between Oct4 and the transactivation domain (TAD) of MyoD (M3O) (see Hirai et al.)

As known in the art, mmRNA may comprise one or more non-natural nucleotides. The nucleoside modification may include a compound selected from the group consisting of pyridin-4-one ribonucleoside, 5-aza-uridine, 2-thio-5-aza-uridine, 2-thiouridine, 4-thio-pseudouridine, 2-thio-pseudouridine, 5-hy-droxyuridine, 3-methyluridine, 5-carboxymethyluridine, 1-carboxymethyl-pseudouridine, 5-propynyl-uridine, 1-propynyl-pseudouridine, 5-taurinomethyluridine, 1-taurinomethyl-pseudouridine, 5-taurinomethy1-2-thiouridine, 1-taurinomethy1-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine. 2-thio-dihydrouridine, 2-thiodihydropseudouridine, 2-methoxyuridine, 2-methoxy-4-thiouridine, 4-methoxy-pseudouridine, 4-methoxy-2-thio-pseudouridine, 5-azacytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyadenosine, 2-methylthio-N-6-(cis-hydroxyisopentenyl) adenosine, N6-glycinylcarbamoyladenosine, N6-threonyl-carbamoyladenosine, 2-methylthio-N6-threonyl carbamoyladenosine, N6.N6-dimethyladenosine, 7-methyladenine, 2-methylthio-adenine, 2-methoxy-adenine, inosine, 1-me-thyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylgua-nosine, N2,N2-dimethylguanosine, 8-oxo-guanosine, 7-me-thy1-8-oxo-guanosine, 1-methyl-6-thio-guanosine, N2-methy1-6-thio-guanosine, and N2,N2-dimethy1-6-thio-guanosine.

In some embodiments, the modifications are independently selected from the group consisting of 5-methylcytosine, pseudouridine and 1-methylpseudouridine. Two modifications of the nucleic acid molecule may be located on nucleosides of the modified nucleic acid molecule. The modified nucleosides may be selected from 5-methylcytosine and pseudouridine.

A 5′ cap structure can be a native 7-methylguanylate cap, or a cap analog, for example anti-reverse cap analog (ARCA), 3″-O-Me-m7G(5′)ppp(5′)G, (m7G(5′)ppp(5′)G), Cap0, Cap1, inosine, N1-methyl-guanosine, 2′ fluoro-guanosine, 7-deaza-guanosine, 8-oxo-guanosine, 2-amino-guanosine, LNA-guanosine, 2-azido-guanosine, etc.

Reprogramming factors, as used herein, refers to one or a cocktail of biologically active polypeptides that act on a cell to alter transcription, and which upon expression, reprogram a somatic cell a different cell type, or to multipotency or to pluripotency. For the purposes of the present invention, the reprogramming factors are provided as mmRNA encoding the polypeptides described below.

In some embodiments the reprogramming factor is a transcription factor, including without limitation, M3O; Sox2; Klf4; c-Myc; and Nanog. Also of interest as a reprogramming factor is Lin28, which is an mRNA-binding protein thought to influence the translation or stability of specific mRNAs during differentiation.

A mmRNA composition may encode one or more biologically active reprogramming factors. The composition may comprise at least about 10 ng/μl each mmRNA specificity, (i.e. encoding each reprogramming factor), at least about 50 ng/μl; at least about 100 ng/μl, at least about 200 ng/μl, at least about 250 ng/μl, at least about 300 ng/μl, or more.

A Klf4 polypeptide is a polypeptide comprising the amino acid sequence that is at least 70% identical to the amino acid sequence of human Klf4, i.e., Kruppel-Like Factor 4 the sequence of which may be found at GenBank Accession Nos. NP_004226 and NM_004235. Klf4 polypeptides, e.g. those that are at least 70%, 75%, 80%, 85%, 90%, 91%, 92%, 95%, 97%, 99%, or 100% identical to the sequence provided in GenBank Accession No. NM_004235. The nucleic acids that encode them find use as a reprogramming factor in the present invention.

A c-Myc polypeptide is a polypeptide comprising an amino acid sequence that is at least 70% identical to the amino acid sequence of human c-Myc, i.e., myelocytomatosis viral oncogene homolog, the sequence of which may be found at GenBank Accession Nos. NP_002458 and NM_002467. c-Myc polypeptides, e.g. those that are at least 70%, 75%, 80%, 85%, 90%, 91%, 92%, 95%, 97%, 99%, or 100% identical to the sequence provided in GenBank Accession No. NM_002467. The nucleic acids that encode them find use as a reprogramming factor in the present invention.

A Nanog polypeptide is a polypeptide comprising an amino acid sequence that is at least 70% identical to the amino acid sequence of human Nanog, i.e., Nanog homeobox, the sequence of which may be found at GenBank Accession Nos. NP_079141 and NM_024865. Nanog polypeptides, e.g. those that are at least 70%, 75%, 80%, 85%, 90%, 91%, 92%, 95%, 97%, 99%, or 100% identical to the sequence provided in GenBank Accession No. NM_024865. The nucleic acids that encode them find use as a reprogramming factor in the present invention.

A Lin-28 polypeptide is a polypeptide comprising an amino acid sequence that is at least 70% identical to the amino acid sequence of human Lin-28, i.e., Lin-28 homolog of C. elegans, the sequence of which may be found at GenBank Accession Nos. NP_078950 and NM_024674. Lin-28 polypeptides, e.g. those that are at least 70%, 75%, 80%, 85%, 90%, 91%, 92%, 95%, 97%, 99%, or 100% identical to the sequence provided in GenBank Accession No. NM_024674. The nucleic acids that encode them find use as a reprogramming factor in the present invention.

An Oct3/4 polypeptide is a polypeptide comprising an amino acid sequence that is at least 70% identical to the amino acid sequence of human Oct 3/4, also known as Homo sapiens POU class 5 homeobox 1 (POU5F1) the sequence of which may be found at GenBank Accession Nos. NP_002692 and NM_002701. Oct3/4 polypeptides, e.g. those that are at least 70%, 75%, 80%, 85%, 90%, 91%, 92%, 95%, 97%, 99%, or 100% identical to the sequence provided in GenBank Accession No. NM_002701. The nucleic acids that encode them find use as a reprogramming factor in the present invention.

A Sox2 polypeptide is a polypeptide comprising the amino acid sequence at least 70% identical to the amino acid sequence of human Sox2, i.e., sex-determining region Y-box 2 protein, the sequence of which may be found at GenBank Accession Nos. NP_003097 and NM_003106. Sox2 polypeptides, e.g. those that are at least 70%, 75%, 80%, 85%, 90%, 91%, 92%, 95%, 97%, 99%, or 100% identical to the sequence provided in GenBank Accession No. NM_003106. The nucleic acids that encode them find use as a reprogramming factor in the present invention.

The terms “primary cells”, “primary cell lines”, and “primary cultures” are used interchangeably herein to refer to cells and cell cultures that have been derived from a subject and allowed to grow in vitro for a limited number of passages, i.e. splittings, of the culture. For example primary cultures are cultures that may have been passaged 0 times, 1 time, 2 times, 4 times, 5 times, 10 times, or 15 times, but not enough times go through the crisis stage.

Starting cell population. As used herein, a “starting cell population”, or “initial cell population” refers to a somatic cell, usually a primary, or non-transformed, somatic cell, such as a fibroblast, which undergoes nuclear reprogramming and genetic correction by the methods of the invention. The starting cell population may be of any mammalian species, but particularly including human cells. Sources of starting cell populations include individuals desirous of cellular therapy, individuals having a genetic defect of interest for study, and the like.

In some embodiments, human cells obtained from a subject for the purpose of nuclear reprogramming and genetic correction may be chosen from any human cell type, including fibroblast cells, adipose tissue cells, mesenchymal cells, bone marrow cells, stomach cells, liver cells, epithelial cells, nasal epithelial cells, mucosal epithelial cells, follicular cells, connective tissue cells, muscle cells, bone cells, cartilage cells, gastrointestinal cells, splenic cells, kidney cells, lung cells, testicular cells, nervous tissue cells, etc. In some embodiments, the human cell type is a fibroblast, which may be conveniently obtained from a subject by a punch biopsy. In certain embodiments, the cells are obtained from subjects known or suspected to have a copy number variation (CNV) or mutation of the gene of interest. In other embodiments, the cells are from a patient presenting with idiopathic/sporadic form of the disease. In yet other embodiments, the cells are from a non-human subject. The cells are then reprogrammed and genetically corrected, and may be subsequently differentiated to adopt a specific cell fate, such as endodermal cells, neuronal cells, for example dopaminergic, cholinergic, serotonergic, GABAergic, or glutamatergic neuronal cell; pancreatic cells, e.g. islet cells, muscle cells including without limitation cardiomyocytes, hematopoietic cells, and the like.

Gene correction. Class 2 Clustered Regularly Interspaced Short Palindromic Repeat (CRISPR) systems, which form an adaptive immune system in bacteria, have been modified for genome engineering. Engineered CRISPR systems contain two components: a guide RNA (gRNA or sgRNA) and a CRISPR-associated endonuclease (Cas protein). The gRNA is a short synthetic RNA composed of a scaffold sequence necessary for Cas-binding and a user-defined ˜20 nucleotide spacer that defines the genomic target to be modified. Thus, one can change the genomic target of the Cas protein by simply changing the target sequence present in the gRNA. The Cas9 protein and the gRNA form a ribonucleoprotein complex through interactions between the gRNA scaffold and surface-exposed positively-charged grooves on Cas9. Cas9 undergoes a conformational change upon gRNA binding that shifts the molecule from an inactive, non-DNA binding conformation into an active DNA-binding conformation. Importantly, the spacer region of the gRNA remains free to interact with target DNA.

Any genomic sequence of about 20 nucleotides can be targeted for protection, provided the sequence is unique compared to the rest of the genome and target is present immediately adjacent to a Protospacer Adjacent Motif (PAM). The PAM sequence serves as a binding signal for Cas9, but the exact sequence depends on which Cas protein is used. Cas9 will only cleave a given locus if the gRNA spacer sequence shares sufficient homology with the target DNA. Once the Cas9-gRNA complex binds a putative DNA target, the seed sequence (8-10 bases at the 3′ end of the gRNA targeting sequence) will begin to anneal to the target DNA. If the seed and target DNA sequences match, the gRNA will continue to anneal to the target DNA in a 3′ to 5′ direction. Thus, mismatches between the target sequence in the 3′ seed sequence completely abolish target cleavage, whereas mismatches toward the 5′ end distal to the PAM often still permit target cleavage.

Cas9 undergoes a second conformational change upon target binding that positions the nuclease domains, called RuvC and HNH, to cleave opposite strands of the target DNA. The end result of Cas9-mediated DNA cleavage is a double-strand break (DSB) within the target DNA (˜3-4 nucleotides upstream of the PAM sequence). The resulting DSB is then repaired by one of two general repair pathways:

In addition to optimizing gRNA design, CRISPR specificity can also be increased through modifications to Cas9. High fidelity Cas9's include eSpCas9(1.1) and SpCas9-HF1. eSpCas9(1.1) contains alanine substitutions that weaken the interactions between the HNH/RuvC groove and the non-target DNA strand, preventing strand separation and cutting at off-target sites. Similarly, SpCas9-HF1 lowers off-target editing through alanine substitutions that disrupt Cas9′s interactions with the DNA phosphate backbone. Another high fidelity Cas9, HypaCas9, contains mutations in the REC3 domain that increase Cas9 proofreading and target discrimination. All three high fidelity enzymes generate less off-target editing than wild type Cas9.

A DNA repair template containing the desired sequence is delivered into the cell type of interest with the gRNA(s) and Cas9. The repair template contains the desired edit as well as additional homologous sequence immediately upstream and downstream of the target (termed left & right homology arms.) The length of each homology arm is dependent on the size of the change being introduced, with larger insertions requiring longer homology arms. The repair template may be a single-stranded oligonucleotide, double-stranded oligonucleotide, or a double-stranded DNA plasmid.

The term “efficiency of reprogramming and genetic correction” may be used to refer to the ability of a cells to give rise to iPS cell colonies when contacted with CRISPR/cas9 for genetic correction in a single step method with reprogramming factors. The efficiency of reprogramming with the methods of the invention vary with the particular combination of somatic cells, method of introducing reprogramming factors, and method of culture following induction of reprogramming.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” are used interchangeably herein and refer to any mammalian subject for whom diagnosis, treatment, or therapy is desired, particularly humans.

Keratinocytes. Keratinocyte cells are found in the deepest basal layer of the stratified epithelium that comprises the epidermis, and are sometimes referred to as basal cells or basal keratinocytes. It is known that 95% of the cells in the epidermis are keratinocytes. Squamous keratinocytes are also found in the mucosa of the mouth and esophagus, as well as the corneal, conjunctival and genital epithelia.

Keratinocytes are maintained at various stages of differentiation in the epidermis and are responsible for forming tight junctions with the nerves of the skin. They also keep Langerhans cells of the epidermis and lymphocytes of the dermis in place.

Keratinocyte stem cells reside in the basal layer of the epidermis, which is the lowest layer of the stratified epithelia. These cells divide to give rise to transient amplifying cells which divide further, and differentiate, as they move upwards in the epidermis. The differentiating cells produce compounds and other proteins which are critical to the integrity of the outermost layer of the skin, the stratum corneum. The keratinocytes in the stratum corneum are dead squamous cells that are no longer multiplying. Once keratinocytes reach the corneum, they are said to be keratinazed, or cornified, creating the tough outer layer of skin.

Induced Keratinocytes, or iKCs, are keratinocyte stem cell-like cells that resemble those that divide to give rise to the different layers of the stratified epidermis. They derive from pluripotent cells (ES or iPS) through the addition of developmental morphogens such as RA and BMP, and like somatic keratinocyte stem cells, can provide a skin barrier when grafted into a wound.

The major proteins found in keratinocytes are keratins. These proteins form the cytoskeleton of keratinocytes, and keratin expression changes as transient amplifying cells differentiate and move to the most superficial stratum corneum.

Selection for keratinocyte stem cells. Following reprogramming, genetic correction and differentiation to keratinocytes, the graftable keratinocytes may be selected for CD49f high expression. The CD49f (ITGA6) protein product is the integrin alpha chain alpha 6. The reference sequence for the human protein may be accessed at Genbank, NP_000201.

Coupling Efficiency (CE) refers to the ratio of the graftable iKCs (CD49f-bright) to corrected iPS at the start of the differentiation process. This ratio is useful to measure the efficiency of the manufacturing process.

Expansion Coefficient (EC) refers to the number of graftable iKCs present after serial passaging compared to the original number of iKCs in the Clinimacs purification. This manufacturing measurement helps define the how many doublings occur after purification.

Selection for cells may use conventional methods. Cells of interest, i.e. cells expressing the marker of choice, may be enriched for, that is, separated from the rest of the cell population, by a number of methods that are well known in the art. For example, flow cytometry, e.g. fluorescence activated cell sorting (FACS), may be used to separate the cell population based on the intrinsic fluorescence of the marker, or the binding of the marker to a specific fluorescent reagent, e.g. a fluorophor-conjugated antibody, as well as other parameters such as cell size and light scatter. In other words, selection of the cells may be effected by flow cytometry. Although the absolute level of staining may differ with a particular fluorochrome and reagent preparation, the data can be normalized to a control. To normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining above the brightness of an isotype matched control, but are not as intense as the most brightly staining cells normally found in the population. An alternative control may utilize a substrate having a defined density of marker on its surface, for example a fabricated bead or cell line, which provides the positive control for intensity.

Other methods of separation, i.e. methods by which selection of cells may be effected, based upon markers include, for example, magnetic activated cell sorting (MACS), immunopanning, and laser capture microdissection.

The affinity reagents may be specific receptors or ligands for the cell surface molecules indicated above. In addition to antibody reagents, peptide-MHC antigen and T cell receptor pairs may be used; peptide ligands and receptors; effector and receptor molecules, and the like. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

Of particular interest is the use of antibodies as affinity reagents. Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The antibodies are added to a suspension of cells, and incubated for a period of time sufficient to bind the available cell surface antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated will be any medium that maintains the viability of the cells. A preferred medium is phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc.

The compositions and methods described here are useful in treating genetic skin disease (genodermatoses). Genodermatoses are genetic diseases with cutaneous expression. They are various (around 400) and almost all rare. Their prevalence is between 1:6000 and 1:500 000. They usually break out at birth or early in life and severely affect children. They are rare and sometimes life-threatening diseases. They have a high impact on the quality of life of the patients and of their family and on the society: social exclusion, disability, short life expectancy. Examples that may be treated with the methods herein include, for example, Ectodermal Dysplasia; Hypohidrotic ectodermal dysplasia (ED1 is the causative gene); Hidrotic ectodermal dysplasia (GJB6 is the causative gene); White sponge nevus (keratin-4 or keratin-13 is the causative gene); Hereditary, benign, intraepithelial-dyskeratosis (a segment of DNA localized at 4q35 is duplicated resulting in triple alleles for 2 linked markers); Pachyonychia congenita (specific mutations in the keratin 16 gene-Jadassohn-Lewandowsky type, mutations of the keratin 17 gene are associated with the Jackso-Lawler form); Dyskeratosis congenita (X-linked recessive mutations in the DKC1 genes); Xeroderma pigmentosum (cause by one of several defects in the excision repair and/or postreplication repair mechanism of DNA); Incontinentia pigmenti (X-linked dominant); Keratosis follicularis (mutation of the gene that encodes intracellular calcium pump ATP2A2); Warty dyskeratoma; Peutz-Jeghers syndrome (LKB1/STK11 mutation); Ehlers-Danlos syndrome; Tuberous sclerosis (TSC-1 and TSC-2); Epidermolysis bullosa; etc.

In one embodiment a condition for treatment is Netherton syndrome, which is a disorder that affects the skin, hair, and immune system. Newborns with Netherton syndrome have skin that is red and scaly (ichthyosiform erythroderma), and the skin may leak fluid. Some affected infants are born with a tight, clear sheath covering their skin called a collodion membrane. This membrane is usually shed during the first few weeks of life. Because newborns with this disorder are missing the protection provided by normal skin, they are at risk of becoming dehydrated and developing infections in the skin or throughout the body (sepsis), which can be life-threatening. Affected babies may also fail to grow and gain weight at the expected rate (failure to thrive). The health of older children and adults with Netherton syndrome usually improves, although they often remain underweight and of short stature.

Netherton syndrome is caused by mutations in the SPINK5 gene, which encodes LEKT1 protein. LEKT1 is a serine peptidase inhibitor found in the skin and in the thymus. LEKT1 controls the activity of serine peptidases in the epidermis, particularly the stratum corneum. Serine peptidase enzymes are involved in normal skin shedding by helping to break the connections between cells of the stratum corneum. LEKT1 is also involved in normal hair growth, the development of lymphocytes in the thymus, and the control of peptidases that trigger immune system function.

Epidermolysis Bullosa. Conditions of interest for treatment with engineered keratinocytes include, without limitation, various forms of Epidermolysis Bullosa, including acquired and congenital forms, the latter of which may be recessive or dominant. Currently classified into four main subtypes (EB simplex, junctional EB, dystrophic EB, and Kindler syndrome, mainly based on the level of skin cleavage), the spectrum of EB extends to more than 30 clinical subtypes with pathogenic mutations in at least 18 distinct genes. Three recent additions to variants of EB are autosomal recessive, and result from mutations in either DST-e (coding for epidermal dystonin, also known as the 230 kDa bullous pemphigoid antigen, BP230), EXPH5 (coding for exophilin-5, also known as Slac2-b), or ITGA3 (coding for the integrin alpha-3 subunit).

Dystrophic Epidermolysis Bullosa (DEB) includes three subtypes: recessive DEB, severe generalized (RDEB-sev gen) (formerly called Hallopeau-Siemens type (RDEB-HS); recessive DEB, generalized other (RDEB-O) (formerly called non-Hallopeau-Siemens type (RDEB-non-HS); and dominant DEB (DDEB). In RDEB-sev gen, blisters affecting the whole body may be present in the neonatal period. Oral involvement may lead to mouth blistering, fusion of the tongue to the floor of the mouth, and progressive diminution of the size of the oral cavity. Esophageal erosions can lead to webs and strictures that can cause severe dysphagia. Consequently, severe nutritional deficiency and secondary problems are common. Corneal erosions can lead to scarring and loss of vision. Blistering of the hands and feet followed by scarring fuses the digits into “mitten” hands and feet, a hallmark of this disorder. The lifetime risk of aggressive squamous cell carcinoma is over 90%. In DDEB, blistering is often mild and limited to hands, feet, knees, and elbows, but nonetheless heals with scarring. Dystrophic nails, especially toenails, are common and may be the only manifestation of DDEB.

Conventional treatment of manifestations is primarily supportive, including wound dressing and nutritional support. Occupational therapy may help prevent hand contractures. Surgical release of fingers often needs to be repeated.

Keratinocytes engineered to express wild-type C7 can find use in therapy for Dystrophic Epidermolysis Bullosa.

In addition to inherited forms of EB, the acquired form of Epidermolysis Bullosa (EBA) involves pathology in type VII collagen and may be treated with the engineered keratinocytes of the disclosure. Circulating autoantibodies in patients with EBA recognize epitopes in type VII collagen molecules, and molecular cloning of the type VII collagen cDNAs have provided the tools to identify the predominant immunoepitopes within the amino-terminal NC-1 domain of type VII collagen. The antigenic properties of the NC-1(VII) domain are further highlighted by the fact that monoclonal antibodies, such as H3A and L3D, which are in clinical use to map type VII collagen in the skin of patients with inherited forms of EB, also identify epitopes in this portion of the protein. In addition to circulating autoantibodies recognizing type VII collagen epitopes in EBA, bullous lesions in some patients with systemic lupus erythematosus have also been associated with anti-type VII collagen antibodies.

Collagen. As used herein the term “collagen” refers to compositions in which at least about 50%, at least about 60%, at least about 70%, at least about 80%, at least about 90%, at least about 95%, or more of the protein present is collagen in a triple helical configuration. The folding of the individual α-chains into the triple-helical conformation is predicated upon the characteristic primary sequence, including repeating Gly-X-Y triplet sequences. Collagens are widely found in vertebrate species and have been sequenced for many different species. Due to the high degree of sequence similarity between species, collagen from different species can be used for biomedical purposes, e.g., between mammalian species, although the human protein may be preferred.

FACIT collagens (fibril-associated collagens with interrupted triple helices) include types IX, XII, XIV, XIX, XX, and XXI. Several of the latter types of collagens associate with larger collagen fibers and serve as molecular bridges, stabilizing the organization of the extracellular matrix. Collagen VII, (COL7A1, Chromosome 3, NC_000003.10 (48576510..48607689, complement)) is of particular interest. Type VII collagen is a major component of anchoring fibrils.

Type VII collagen is a long, 424 nm, triple-helical domain with flanking non-collagenous sequences. Type VII collagen molecules include a central collagenous, triple-helical segment flanked by the non-collagenous NC-1 and NC-2 domains. Unlike interstitial collagens, the repeating Gly-X-Y sequence is interrupted by 19 imperfections due to insertions or deletions of amino acids in the Gly-X-Y repeat sequence. Most notably, in the middle of the triple-helical domain, there is a 39-amino acid non-collagenous “hinge” region which is susceptible to proteolytic digestion with pepsin. The amino-terminal NC-1 domain of type VII, approximately 145 kDa in size, includes sub-modules with homology to known adhesive proteins, including segments with homology to cartilage matrix protein (CMP), nine consecutive fibronectin type III-like (FN-III) domains, a segment with homology to the A domain of von Willebrand factor, and a short cysteine and proline-rich region. The carboxy-terminal non- collagenous domain, NC-2, is relatively small, ˜30kDa, and it contains a segment with homology to Kunitz protease inhibitor molecule.

The human type VII collagen gene, COL7A1 has a complex structure with a total of 118 separate exons. The gene is, however, relatively compact, and most of the introns are relatively small; consequently, the size of the entire human COL7A1 gene is only ˜32 kb, encoding a messenger RNA of ˜8.9 kb. COL7A1 has been mapped to the short-arm of human chromosome 3, region 3p21.1. The type VII collagen gene structure and the encoded primary sequence of the protein are well conserved, and for example, the mouse gene shows 84.7% homology at the nucleotide and 90.4% identity at the protein level.

Type VII collagen is synthesized both by epidermal keratinocytes and dermal fibroblasts in culture. Upon synthesis of complete pro-α1(VII) polypeptides, three polypeptides associate through their carboxy-terminal ends to a trimer molecule which in its collagenous portion folds into the triple-helical formation. The triple-helical molecules are then secreted to the extracellular milieu where two types of VII collagen molecules align into an anti-parallel dimer with the amino-terminal domains present at both ends of the molecule. This dimer assembly is accompanied by proteolytic removal of a portion of the carboxy-terminal end of both type VII collagen molecules and stabilization by inter-molecular disulfide bond formation. Subsequently, a large number of these anti-parallel dimers aggregate laterally to form anchoring fibrils.

Glycine substitution mutations in the triple helical domain of COL7A1 (especially in exons 73, 74, and 75) predominate in Dominant Dystrophic Epidermolysis Bullosa (DDEB). Mutations p.Gly2034Arg and p.Gly2043Arg are the most common DDEB-causing mutations, making up 50% of the dominant mutations reported in the largest US cohort. Glycine substitutions as well as other amino acid substitutions and splice junction mutations outside of this region may also be found in dominant DEB.

More than 400 recessive DEB-causing mutations spanning the entire gene have been described for all forms of DEB. Each mutation, however, accounts for no more than 1%-2% of the total number of mutations. Null mutations predominate in RDEB, though glycine substitutions and other amino acid substitutions have been described. Milder forms of RDEB are often caused by splice junction mutations or other missense mutations.

A “native sequence” polypeptide is one that has the same amino acid sequence as a polypeptide derived from nature. Such native sequence polypeptides can be produced by recombinant means according to the methods set forth herein. Thus, a native sequence polypeptide can have the amino acid sequence of, e.g., naturally occurring human polypeptide, murine polypeptide, or polypeptide from any other mammalian species, and the like. The term “native sequence collagen VII protein” includes the native proteins with or without the initiating N-terminal methionine (Met).

A “variant” polypeptide means a biologically active polypeptide as defined below having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active collagen VII variant will have an amino acid sequence having at least about 90% amino acid sequence identity with a native sequence collagen VII polypeptide, preferably at least about 95%, more preferably at least about 99%.

A “functional derivative” of a native sequence collagen, VII polypeptide is a compound having a qualitative biological property in common with a native sequence collagen VII polypeptide. “Functional derivatives” include, but are not limited to, fragments of a native sequence and derivatives of a native sequence collagen VII polypeptide and its fragments, provided that they have a biological activity in common with a corresponding native sequence collagen VII polypeptide. The term “derivative” encompasses both amino acid sequence variants of collagen VII polypeptide and covalent modifications thereof.

The term “wound bed” refers to the uppermost viable layer of wound. In one embodiment, the wound bed is covered by slough or eschar. In another embodiment, the wound bed can be assessed for presence of granulation tissue fibrin slough, eschar, bone, tendon, and/or other underlying structures.

The term “genetic modification” refers to a process of altering a gene of an organism or inserting a gene from one organism into another organism. In one embodiment, the genetic modification comprises, consists essentially of, or yet consists of insertion, deletion, and/or mutation. The term “insertion” means addition of one or more nucleotide base pairs into a nucleotide sequence. The term “deletion” refers to a part of a chromosome or a nucleotide sequence that is removed or missing. The term “mutation” is alteration of nucleotide sequence (e.g., DNA sequence). The mutation can occur in various sizes, including, but not limited to, a single base pair (i.e., point mutation), several base pairs, or up to a large segment of chromosome.

The term “conservative genetic modification” refers to genetic modification that maintain same or similar biochemical properties of a polypeptide encoded by the genetically modified gene. For example, both aspartic acid and glutamic acid are both small, negatively charged residues. In some embodiment, it is a conservative genetic modification by mutate aspartic acid to glutamic acid in a polypeptide.

### Methods

A starting population of somatic cells, e.g. fibroblasts, are genetically corrected at a locus of interest, and reprogrammed to pluripotency in a single step for GMP compatible integration-free, xeno-free, feeder-free iPSC, which iPSC can provide a source of further differentiated cells for treatment of genetic disorders. Engineering iPSC allows patient samples to be obtained from cells such as fibroblasts, which may be more available than cells from affected tissues. Further, the iPSC samples can be stored frozen for long periods of time, and expanded in suitable numbers for multiple administrations of a tissue graft.

The starting population of somatic cells, conveniently fibroblasts although other cell types also find use, are genetically corrected by CRISPR technology as described above. After a period of from about 2 to about 6 days, the cells are contacted with a population of mmRNA encoding reprogramming factors, as defined above, in a combination and quantity sufficient to reprogram the cell to pluripotency. Reprogramming factors may be provided to the somatic cells individually or as a single composition, that is, as a premixed composition, of reprogramming factors. Repeated transfections are typically required, at least 2, at least 3, at least 4, at least 5, at least 6, at least 7, at least 8, at least 9, at least 10 transfections, which can be repeated daily, seemi-daily, every two days, etc. In some embodiments, a set of at least three mRNA encoding reprogramming factors is added, e.g., Oct3/4 or a modified variant thereof, Sox2, and Klf4, c-myc, nanog or 1in28. In some embodiments, a set of five mRNA encoding reprogramming factors is provided to the cells e.g., Oct4 fused with the MyoD transactivation domain (called M3O), Sox2, Klf4, cMyc, and Lin28A.

Methods for introducing the mmRNA encoding reprogramming factors to somatic cells include transfection, lipofection, electroporation, exosomal delivery and the like, as known in the art. Following introduction of the mmRNA, cells are incubated for about 30 minutes to about 72 hours, e.g., 2 hours, 4 hours, 8 hours, 12 hours, 18 hours, 24 hours 36 hours, 48 hours, 60 hours, 72 hours, or any other period from about 30 minutes to about 72 hours. The reprogramming factors may be provided to the subject cells for about one to about 4 weeks, e.g. from about two to about 3 weeks.

The dose of mmRNA encoding reprogramming factors will vary with the nature of the cells, the factors, the culture conditions, etc. In some embodiments the dose will be from about 1 nM to about 1 μM for each factor, more usually from about 10 nM to about 500 nM, or around about 100 to 200 nM. In embodiments where an additional activator is used, the cells are initially exposed during exposure to the reprogramming actors for at least about 1 day, at least about 2 days, at least about 4 days, at least about 6 days or one week, and may be exposed for the entire reprogramming process, or less. The dose will depend on the specific agonist, but may be from about 1 ng/ml to about 1 μg/ml, from about 10 ng/ml to about 500 ng/ml.

Following introduction of reprogramming factors, the somatic cells may be maintained in a culture medium in the absence of feeder layers, i.e. lacking somatic cells other than those being induced to pluripotency. Feeder layer free cultures may utilize a protein coated surface, e.g. matrigel, etc. The medium may also be free of xenogeneic (non-human) protein factors such as fetal bovine serum.

iPS cells induced to become such by the methods of the invention have an hESC-like morphology, growing as flat colonies with large nucleo-cytoplasmic ratios, defined borders and prominent nuclei. In addition, the iPS cells may express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, the iPS cells are capable of forming teratomas. In addition, they are capable of forming or contributing to ectoderm, mesoderm, or endoderm tissues in a living organism.

The iPS cells produced by the above methods may be used for reconstituting or supplementing differentiating or differentiated cells in a recipient. The induced cells may be differentiated into cell-types of various lineages. Examples of differentiated cells include any differentiated cells from ectodermal (e.g., neurons and fibroblasts), mesodermal (e.g., cardiomyocytes), or endodermal (e.g., pancreatic cells) lineages. The differentiated cells may be one or more: pancreatic beta cells, neural stem cells, neurons (e.g., dopaminergic neurons), oligodendrocytes, oligodendrocyte progenitor cells, hepatocytes, hepatic stem cells, astrocytes, myocytes, hematopoietic cells, or cardiomyocytes.

There are numerous methods of differentiating the induced cells into a more specialized cell type. Methods of differentiating induced cells may be similar to those used to differentiate stem cells, particularly ES cells, MSCs, MAPCs, MIAMI, hematopoietic stem cells (HSCs). In some cases, the differentiation occurs ex vivo; in some cases the differentiation occurs in vivo.

The induced cells, or cells differentiated from the induced cells, may be used as a therapy to treat disease (e.g., a genetic defect). The therapy may be directed at treating the cause of the disease; or alternatively, the therapy may be to treat the effects of the disease or condition. The induced cells may be transferred to, or close to, an injured site in a subject; or the cells can be introduced to the subject in a manner allowing the cells to migrate, or home, to the injured site. The transferred cells may advantageously replace the damaged or injured cells and allow improvement in the overall condition of the subject. In some instances, the transferred cells may stimulate tissue regeneration or repair.

The transferred cells may be cells differentiated from induced cells. The transferred cells also may be multipotent stem cells differentiated from the induced cells. In some cases, the transferred cells may be induced cells that have not been differentiated.

The number of administrations of treatment to a subject may vary. Introducing the induced and/or differentiated cells into the subject may be a one-time event; but in certain situations, such treatment may elicit improvement for a limited period of time and require an on-going series of repeated treatments. In other situations, multiple administrations of the cells may be required before an effect is observed. The exact protocols depend upon the disease or condition, the stage of the disease and parameters of the individual subject being treated.

The cells may be introduced to the subject via any of the following routes: parenteral, intravenous, intraarterial, intramuscular, subcutaneous, transdermal, intratracheal, intraperitoneal, or into spinal fluid.

The differentiated cells may be administered in any physiologically acceptable medium or device. They may be provided alone or with a suitable substrate or matrix, e.g. to support their growth and/or organization in the tissue to which they are being transplanted. Usually, at least 1×10⁵ cells will be administered, preferably 1×10⁶ or more. The cells may be introduced by injection, catheter, sprayer (j.e. Spray-on-skin) or the like. The cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or stromal cells associated with progenitor cell proliferation and differentiation.

In some embodiments the genetically corrected iPSC are differentiated to keratinocytes, optionally being selected for CD49f expression prior to manufacture of the final epithelial sheet, and used for treatment of EB. In such methods, a keratinocyte sheet is used as a graft on an EB wound. In one aspect, the wound is free of non-corrected wound bed keratinocytes. In one embodiment, the wound is treated to ablate non-corrected wound bed keratinocytes. In another aspect, the subject suffers from Recessive Dystrophic Epidermolysis Bullosa (RDEB). In a different aspect, the subject is human.

In some embodiments, the keratinocyte sheet is placed on an acellular matrix, a collagen matrix, or a biocompatible mesh. In one embodiment, the biocompatible mesh is made of thermoplastic resin, polyethylene, ultra-high molecular weight polyethylene, high molecular weight polyolefin, uncoated monofilament polypropylene, polyether ether ketone, polyethylene terephthalate, polytetrafluoroethylene, expanded polytetrafluoroethylene, nylon, silicon, or any combination thereof. In some embodiments, the skin cells comprise, alternatively consist essentially of, or yet further consist of keratinocytes.

In one aspect, the biocompatible mesh can be made from non-resorbable materials, including, but not limited to, biocompatible metals such as titanium alloys, stainless steel, cobalt-chromium alloys, and nickel-titanium alloys. In another aspect, the layer of biocompatible mesh can be made from non-resorbable polymeric materials, including, but not limited to, thermoplastic resins, polyethylenes, ultra-high molecular weight polyethylene, high molecular weight polyolefins, uncoated monofilament polypropylene, polyether ether ketone, polyethylene terephthalate, polytetrafluoroethylene, expanded polytetrafluoroethylene, nylon, any polymer or aliphatic hydrocarbons containing one or more double bonds, any other appropriate porous materials, or any other appropriate porous material that can be bent or otherwise formed into a shape.

In another aspect, the biocompatible mesh can be composed of a synthetic or biological resorbable polymeric material, including, but not limited to, polyglycolic acid, poly-L-lactic acid (PLLA), poly-D,L-lactic acid (PDLA), trimethylene carbonate (TMC), poly-£-caprolactone, poly-P-dioxanone, copolymers of lactide and glycolide (PLGA), polyhydroxy-3-butyrate, collagen, hyaluronic acid, silk, biocellulose, other protein-based polymers, polysaccharides, poly(DTE carbonate), polyarylates, blends of PLLA, PLDA, or PLGA with TMC and other combinations of these polymers.

In one embodiment, the biocompatible mesh is made of thermoplastic resin, polyethylene, ultra-high molecular weight polyethylene, high molecular weight polyolefin, uncoated monofilament polypropylene, polyether ether ketone, polyethylene terephthalate, polytetrafluoroethylene, expanded polytetrafluoroethylene, nylon, silicon, or any combination thereof.

Also provided in this disclosure is a composition comprising, alternatively consisting essentially of, or yet further consisting of a keratinocyte sheet, wherein the keratinocyte sheet is prepared by a process comprising, alternatively consisting essentially of, or yet further consisting of the steps of: obtaining a population of fibroblasts from a subject; genetically correcting and reprogramming the cells in a single step method; differentiating the reprogrammed cells to form keratinocytes, selecting for expression of CD49f, and culturing the CD49f⁺ cells to form the keratinocyte sheet.

In one embodiment, the functional COL7A1 protein is a full-length wild-type human COL7A1 protein. In one aspect, the functional COL7A1 protein comprises, alternatively consists essentially of, or yet further consists of a genetic modification from a full-length wild-type human COL7A1 protein. In another aspect, the functional COL7A1 protein comprises, alternatively consists essentially of, or yet further consists of a genetic modification from a full-length wild-type human COL7A1 protein, wherein the genetic modification is conservative. In a further aspect, the genetic modification comprises, alternatively consists essentially of, or yet further consists of insertion, deletion, and/or mutation.

In certain embodiments, cells are cultured for 1-21 days. In further embodiments, cells are cultured 7, 14, 21 days or longer. Thus, cells may be cultured under appropriate conditions for 1, 2, 3, 4, 5, 6, 7, 8, 9, 10, 11, 12, 13, 14, 15, 16, 17, 18, 19, 20, 21, 22, 23, 24, 25, 26, 27, 28, 29, or more days. Cells are re-plated, and media and supplements may be added or changed as needed using techniques known in the art.

In certain embodiments, the genetically altered cells may be cultured under conditions and for sufficient time periods such that at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% of the cells express the C7 transgene.

In one embodiment, the cell compositions of the present disclosure comprise, alternatively consist essentially of, or yet further consist of an integration-free genetically altered autologous keratinocyte population, expressing a native human C7 protein in an amount effective for the treatment of EB. Target cell populations are grown in sheets for engraftment onto a subject, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives.

Cell compositions of the present disclosure are administered in a manner appropriate to the treatment of EB. The quantity and frequency of administration will be determined by such factors as the condition of the patient, and the type and severity of the patient's disease, although appropriate dosages may be determined by clinical trials.

The cells may be administered to the subject by methods well known to those of skill in the art, typically in the form of a skin graft. A medical practitioner will be able to determine a suitable administration route for a particular subject based, in part, on the type and location of the disease. The transfected cells may be administered locally to a wound site.

Pharmaceutical preparations of engineered cells for administration to a subject are contemplated by the present invention. One of ordinary skill in the art would be familiar with techniques for administering cells to a subject. Furthermore, one of ordinary skill in the art would be familiar with techniques and pharmaceutical reagents necessary for preparation of these cell sheets prior to administration to a subject.

Pharmaceutical compositions of the present invention comprise an effective amount of a solution of the transfected cells in a pharmaceutically acceptable carrier or aqueous medium. As used herein, “pharmaceutical preparation” or “pharmaceutical composition” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the cells, its use in the therapeutic compositions is contemplated. Supplementary active ingredients can also be incorporated into the compositions. For human administration, preparations should meet sterility, pyrogenicity, general safety, and purity standards as required by the FDA Center for Biologics.

A person of ordinary skill in the art would be familiar with techniques for generating sterile solutions for application by any other route. Determination of the size of the cell graft and the number of cells on the graft will be made by one of skill in the art. In certain aspects, multiple doses may be administered over a period of days, weeks, months, or years. A subject may receive, for example, 1, 2, 3, 4, 5, 6, 7, 8, 9, 10, 11, 12, 13, 14, 15, 16, 17, 18, 19, or 20 pieces of graft in the same area or a different area. In one embodiment, the subject may be re-grafted in the same area or a different area. In another embodiment, the subject's biological sample (e.g., keratinocytes or corneal cells) is stored in proper conditions. Once the biological sample is stored, no punch biopsy is necessary if the subject requires a new graft. The stored biological samples can provide sufficient or supplemental information to determine the graft needed by the subject.

When “an effective amount” or “therapeutic amount” is indicated, the precise amount of the compositions of the present disclosure to be administered can be determined by a physician with consideration of individual differences in age, weight, and condition of the patient (subject). It can generally be stated that a cell composition comprising the cells described herein may be administered in the amount of 1-100, 1-10³, 1-10⁴, 1-10⁵, 1-10⁶, 1-10⁷, or more than 10⁷ cells, including all integer values within those ranges. Cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments of the present disclosure, keratinocytes that are genetically engineered using the methods described herein, or other methods known in the art, are administered to a patient in conjunction with (e.g., before, simultaneously, or following) any number of relevant treatment modalities or delivery devices.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

RDEB patient keratinocytes contain mutations in the COL7A1 locus, a gene encoding for type VII collagen, the main component of the anchoring fibrils which tether the epidermis to the dermal tissue underneath. From birth, RDEB patients suffer profound skin fragility and an association with delayed wound healing and persistent erosions. The therapeutic strategy is that COL7A1-corrected autologous keratinocyte stem cell sheets, when grafted onto wounds, adhere tightly and provide long-term wound closure. iPSCs can be grown in large quantities and be induced to differentiate into iKCs. Clinical scaling and manufacturing of patient iKC sheets can be achieved by differentiating patient-derived, COL7A1-corrected iPSCs.

Because of severe depletion of epidermal stem cells that prevent scalability, we developed an alternative strategy to amplify autologous, COL7A1-corrected keratinocyte stem cells called iPSC-derived RDEB Cell Therapy. Starting with patient fibroblasts, DEBCT manufacturing passes through 4 Process Intermediates to generate a final DEBCT graft. Using this process, we have achieved similar results to clinical trials in which RDEB patient-derived iKC grafts are generated, with strong in vivo adhesive properties. Table 1 below outlines the differentiation results from 7 iPSC intermediates derived from 4 patients. In vitro organotypic cultures have been performed on all 5, with mouse grafts completed with one line. The sheets adhered tightly to the dermis, produced type VII collagen, and showed similarity of gene expression with donor keratinocytes. Using the collagen VII antibody, we showed that full-length and functional collagen VII is produced by the iKCs, secreted and deposited into the basement membrane zone. Staining with differentiation markers K14, K10, involucrin, and ITGA6 demonstrate the effective differentiation into a human stratified epidermis. The surface grafts were assayed at 5 weeks. This demonstrates the ability to reproducibility generate functional human stratified epidermal grafts in vitro and in vivo.

TABLE 1
Summary of patients and iPS cell intermediates tested

| | IPSC | | CRISPR | In vitro | In vivo |
|---|---|---|---|---|---|
| Patient | Inter- | | | organotypic | mouse |
| ID | mediate | Genotype | Method | skin | grafts |
| C02 | III.6 | Colorado | RNA | YES | YES |
| | | 7485 + 5G > A | | | |
| DEB135 | S21 | 6781 C > T | RNP | YES | In process |
| DEB135 | R10 | 6781 C > T | RNP | YES | In process |
| DEB135 | R24 | 6781 C > T | RNP | YES | In process |
| DEB134 | 24 | 6781 C > T | RNP | In process | In process |
| DEB125 | 5 | Colorado | RNP | In process | In process |
| | | 7485 + 5G > A | | | |
| C02 | 4 | Colorado | RNP | In process | In process |
| | | 7485 + 5G > A | | | |

Technical improvements include: a) Development of a murine feeder-free, xeno-free, viral-free method of iPS cell reprogramming; b) Development of an integration-free, vector-free, single clonal step for production of the autologous, corrected iPS intermediate, c) Development of a murine feeder-free, xeno-free defined and reproducible method of iKC differentiation from ES/iPSCs; d) identification and use of ITGA6 as a biomarker for the purification of iKCs. These technical advances significantly shorten the manufacturing time and eliminate multiple subcloning steps. Mutations invariably accumulate with time in culture, and subcloning selects for cells with a growth advantage. Therefore, shortening of the manufacturing process and elimination of subcloning makes the cell product safer. In addition, the introduced simplifications make clinical development and patient treatment with iPS cell- derived iKC sheets more easily achievable.

The process comprises 7 steps (M1-7), 4 process intermediates (PI #1-4), and the final product. The process is continuous and does not involve any freezing or banking steps from skin biopsy to the skin grafts. A frozen reserve of PI #1,2, and 4 is maintained, in case a later step fails, so we can go back to those intermediates. DEBCT steps M1 (skin biopsy and primary cell culture) to generate Intermediate #1, and M7 (DEBCT iKC sheet generation and transport to patient) are identical to those in the LEAES (LZRSE-Col7A1 engineered autologous epidermal sheet) protocol. Step M2 (Reprogram and correct donor cells) generates Intermediate #2, step M3 (safely expanding corrected IFS intermediate) generates Intermediate #3, step M4 (Differentiate Intermediate #3 into iKCs) and M5 (Selecting iKCs by CliniMacs) generate Intermediate #4, and step M6 (expansion of iKCs) leads to DEBCT production.

Step M1: Derivation of RDEB fibroblasts from skin biopsy (PI #1). A skin biopsy is minced using scalpels and chunks of tissue are placed in a tissue culture dish. After 5-9 days fibroblasts grow out of the tissue chunks and populate the dish. The media, e.g. DMEM, may be supplemented with 10% FBS. The FBS may be replaced with human serum, HSA, or human platelet lysates. After 8-12 days, the cells are dissociated and subjected to Step M2. In parallel, a small aliquot of fibroblasts is subjected to analysis of release criteria.

Step M2: Integration-free, xeno-free, feeder-free single clonal step production of the autologous, CRISPR-corrected iPSC intermediates (PI #2 and #3) from RDEB fibroblasts (PI #1). Manufacturing of COL7A1-corrected autologous iPSCs is optimized through the development of an integration-free, feeder-free, xeno-free, single clonal step method using preformed ribonuclear protein (RNPs) complexes with high-fidelity CAS9 and guide RNAs and mRNAs encoding the Yamanaka reprogramming factors. The combination of footprint-free reprogramming and gene editing of patient somatic cells into a one-step process is dependent on the ability to efficiently generate iPSCs using non-integration- based methods and the development of high-efficiency gene editing. DNA-based reprogramming methods suffer from safety concerns that DNA may randomly integrate into the genome creating an oncogenic event. We have focused on optimizing reprogramming using synthetic capped mRNAs containing modified nucleobases (modified mRNA). Incorporation of a modified version of Oct4 fused with the MyoD transactivation domain (called M3O), Sox2, Klf4, cMyc, and Lin28A significantly increases the efficiency of reprogramming when combined with a feeder-free system. We have optimized this mRNA reprogramming method using xeno-free media and synthesized mRNAs and observed robust iPS cell colony formation in all patient fibroblast lines tested so far.

To enable a single, cGMP compatible reprogramming and gene repair step, we chose a strategy using clustered regularly interspaced short palindromic repeats (CRISPR)-associated CAS9 nucleases, to induce a precise DNA double-strand break (DSB) at endogenous pathogenic genomic loci with high efficiency. The presence of synthesized exogenous single stranded donor DNA, encoding the correct gene sequence and homology to the region flanking the DSB, triggers homologous recombination (HR)-directed repair of the CRISPR-mediated DSB and replacement of the pathogenic sequence with the corrected one. The advantages of this approach include the lack of integrating viruses or clonal selection, and the ability to perform both reprogramming and mutational correction in one step. We have tested delivering the CAS9 protein by mRNA transfection (mRNA), and by RNP complexes. To minimize off target cutting we use the high-fidelity CAS9 protein (Aldeveron) in RNP complexes. Together, our group has generated a one-step method for reprogramming and CRISPR-based genetic correction of mutations that yields clonal and COL7A1-edited iPSC lines.

After fibroblast are established from the biopsy, they are treated with RNPs and DNA oligonucleotides. Cells were transfected with 10 pmole single stranded (ss) DNA oligo donor of 200 bases length, incubated for 24 h, and transfected again with 10 pmole donor and 7.5 pmole CRISPR/CAS9. Transfection of CAS9 without a sgRNA served as negative controls. After 4 days in culture, cells are reprogrammed by repeated transfections with the mRNAs encoding the Yamanaka factors in defined reprogramming media. iPS cell colonies form within 10 days. About ˜100 colonies are picked into 48 wells and upon confluency all re-grown colonies are characterized by droplet digital PCR to quantify COL7A1 correction of the patient mutation via a reference probe that displays a biallelic locus (e.g. RPP30).

Colonies positive by ddPCR (i.e. range of corrected allele frequency 0.4-0.6 in comparison with biallelic reference probe) are further expanded and the remaining colonies are discarded. Expanded ddPCR+ colonies are subject to in-depth characterization of the COL7A1 locus by PCR amplification, Sanger sequencing of the PCR product, Topo-cloning and Sanger sequencing of 100 individual PCR amplified loci. Those iPSC lines whose genome yields the expected size PCR product of the amplified edited COL7A1 locus, and whose Sanger sequencing of this edited locus and the unedited locus (i.e. the other COL7A1 allele) shows the expected sequence in 95 out of 100 isolated alleles (i.e. Topo clones)are labeled Process Intermediate #2. A fraction is stored as a reserve and the rest is expanded as outlined below.

Step M3: Stable iPS cell expansion to PI #3 in StemFit media. A major problem in the iPSC field is karyotypic (KT) instability during expansion. The standard commercial media available (mTESR, E8 etc.) do not support KT stability in iPSCs over long culture periods. This is likely due to the limited iPSC survival upon splitting, revealing a need for more optimal growth media. A GMP-grade iPSC media that allows iPSC expansion without accumulation of KT abnormalities called “StemFit” in combination with coating the dishes with Laminin 5,1,1 or a fragment of Laminin5,1,1 called E8 fragment performed the best in cell survival and growth and karyotype stability for at least 10 passages. After expansion to 100 million cells, we perform karyotype analysis. The release criteria for PI #3 will be a normal KT. In addition, we will sequence 130 oncogenes using the CLIA-approved STAMP v2 Oncopanel for our own information.

Step M4: Defined, feeder-free, and xeno-free iKC differentiation to PI #4. We have generated a defined and reproducible differentiation assay from PI #3 to iKCs (PI #4) by the addition of retinoic acid (RA) and bone morphogenetic protein (BMP). Three major improvements made in manufacturing of iKCs (PI #4) include: Use of embryoid bodies (EB), identification of a E6 defined media that reduces line-to-line variability, and identification of the correlation of ITGA6 surface expression with epidermal stem cells and graftability allowing CliniMACS purification of iKCs.

A key manufacturing efficiency metric is the ratio of input iPSC (PI #3) to the number of iKCs generated (PI #4), a metric we call Coupling Efficiency (CE). The LEAES product requires plating 5×10⁶ keratinocytes into a rectangular Nunclon D dish for each graft. Our clinical protocol allows application of up to 6 grafts, which would require 30 million iKCs. We performed defined differentiations of PI #3 into PI #4 with 4 independent RDEB iPS lines in multiple runs (>2 each). We found at Day 45 of differentiation, approximately 15% of the culture contained ITGA6 bright cells, a defining marker for iKCs (see below). Overall, this gave us an average CE of approximately 50%.

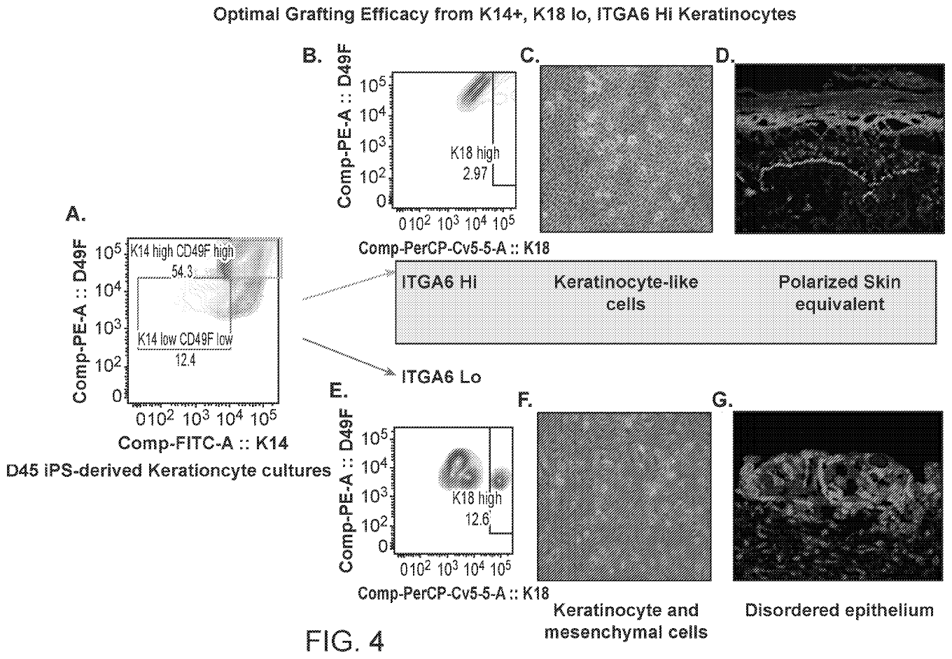

Step M5: Purification of graftable iKCs (PI #4) based on CD49f (ITGA6)^{HI} sorting. Through chromatin dynamic analysis of IPS differentiation, we focused on identifying in-process potency markers that correlate with keratinocyte stem cells and tissue graftability. We found that ITGA6 is one of the earliest surface markers to arise in definitive mature keratinocytes and that ITGA6 bright population correlates with a K14+, K18 - expression basal keratinocyte phenotype. More importantly ITGA6 bright cells displayed enhanced graftability, resulting in an organotypic epidermis that displays proper polarity and differentiation (FIG. 4B-D). By contrast, the unsorted cells contained contaminating immature and undifferentiated cells that were K18 expressing, and generated organotypic cultures with misadherent and mal-oriented keratinocytes. FACS sorting of ITGA6 bright cells and subsequent expansion using CD49f antibodies generate mature iKCs that perform well in organotypic cultures. An alternative cell selection method uses Miltenyi CliniMACS magnetic bead purification system, for example anti-biotin microbeads and Miltenyi biotinylated REA518 humanized anti-ITGA6 antibody.

Step M6: Expansion of selected iKCs after selection in Defined Keratinocyte Media. After selecting early iKCs, we expand iKCs 2-4 passages in keratinocyte defined media DKSFM. We quantify the expansion capabilities of this step by the metric Expansion Coefficient (EC). This media is serum free and low calcium promoting iKC growth and selecting against the growth of other cell types (for example undifferentiated TRA1-60+/SSEA3+ IPSCs). Because we need to produce 30 million iKCs for clinical trial, we have identified selective medias that are GMP-compatible and have favorable ECs. The current defined media has an EC of approximately 10 and we are testing medias that have ECs of 18.

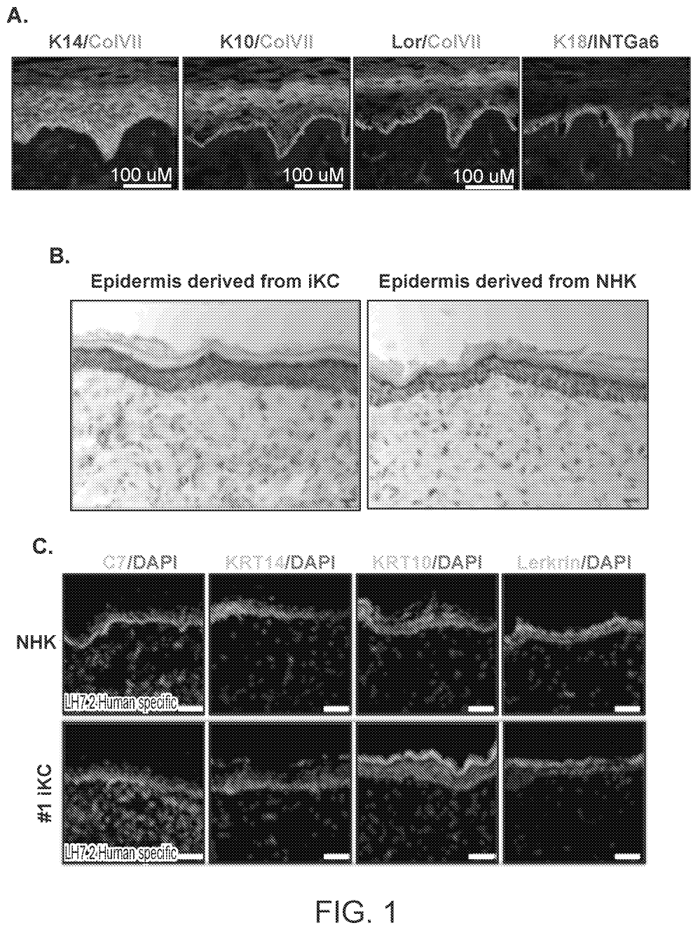

Step M7: Forming the graftable epithelial sheet, the DEBCT final product. We perform a final flow cytometry analysis after in vitro expansion to assess the proportion of iKCs (using CD104, the coreceptor for CD49f), expected fibroblast feeders (CD90), and undifferentiated iPSCs (Tra1-60). We have determined that the product should be >60% CD104, <25% CD90, and <0.1% Tra1-60. With DEBCT defined by cell composition, the final step is the same as the LEAES graft formation and delivery manufacturing step. After expansion, approximately 5 million iKCs are placed in culture, grown to confluence and then released onto Vaseline gauze and delivered to the operating room (see FIG. 1A-C). Wound site selection, graft bed preparation, and grafting of DEBCT onto the patient will follow LEAES IND protocol.

### Example 2

### DEBCT Safety Studies

DEBCT manufacturing requires multiple novel genetic manipulations over a three-month period. We have carried out several assays to demonstrate the genomic integrity and safety of DEBCT. Our major concerns are accumulation of deleterious genetic variations, the presence of undifferentiated pluripotent cells, and the presence of transformed iKCs.

Sanger Sequencing provides sensitive detection of COL7A1 variants. To obtain high-quality information about the genomic region in which we are editing, we endeavored to deep sequence the COL7A1 gene around the mutation region. We accomplished this by ddPCR to look at individual cells for mutation correction, by performing qPCR amplification and then sequencing of the region surrounding the mutation, and by subcloning and sequencing 100 clones and determining the frequency of variants.

No Detectable Mutation Selection in the corrected iPS cell intermediate. An important question was whether our long-term cultures selected for particular mutations. To address this question we performed whole genome and targeted resequencing on three independent primary seed banks, iPSCs, and corrected iPSC lines and determined the mutational burden and identity of recurrent mutations generated. Despite previous reports of p53 and other mutations that have been identified, our sequencing showed a lack of distinct mutation selection during manufacturing. Although we cannot rule out the random occurrence of a deleterious mutation appearing, our data argue against the selection of particular growth or stem cell mutants with our protocol.

Using the Stanford STAMPV2 panel to select DEBCT without deleterious variants. To characterize the genetic variation inevitable in cultured cells and that derive from treatment with CRISPR-Cas9, we have previously published whole genome sequencing of iPSCs, but found the process suffered from our inability to judge the significance of the changes. Given that our main concern is potential squamous cell carcinoma formation (which is also the main cause of lethality of RDEB patients) we reason it to be most relevant to exclude mutations known to cause cancer. We have chosen to focus on sequencing an extensive list of genes whose function is well-characterized in the tumor setting. We propose to use a CLIA-approved test called the Stanford Solid Tumor Actionable Mutation Panel or STAMPV2, that sequences 130 of the most common oncogenes, with a limit of detectability of 5% (Appendix 1). The Stanford Oncopanel provides a reliable and meaningful survey of deleterious variants in the genome. We have completed two sets of patient trios, the fibroblasts (PI #1), the corrected iPS intermediate (PI #3), and the iKCs (DEBCT). In one manufacturing process run, we found NO mutations above the limit of detectability (5%) in any of the intermediates. By contrast, in the other process, while the DPH3 (diphthamide synthesis 3) mutation was not detectable in the polyclonal fibroblast population, in the clonal IPS cell intermediate the allele frequency suggested that every cell was heterozygous for this mutation. Mutations in the promoter of DPH3 have been associated with the basal cell carcinoma form of keratinocyte tumors. We conclude that this particular iPS cell clone carried a preexisting or early occurring mutation and would not be used further. We are in the process of sequencing additional trios of iPS cell intermediates to obtain more information. By eliminating all Intermediates that carry detectable mutations in the STAMP oncopanel we decrease the risk of potential tumor formation from the graft and potentially decreased this risk even below the risk of the patients own skin cells because there is a high chance that a fraction of RDEB patients skin cells carry SCC predisposing mutations.

Efficient differentiation with substantial loss of undifferentiated iPS cells in keratinocyte media, and undetectable Incorporation of undifferentiated iPSCs into DEBCT grafts. Another important safety concern is the potential formation of benign teratomas due to contamination of undifferentiated iPS cells. We have made three key observations showing that the risk of contaminating iPS cells in the manufacturing process and their risk to form teratomas on dermis is exceedingly low. (i) Nearly undetectable Tra1-60 cells at PI #4 stage. We have shown using FACS sorting of Day 42 cultures with an ITGA6 antibody the presence of near undetectable level of IPS cells, 0.3%, of Tra1-60+; ITGA6+ as markers for IPS cells. (ii) Substantial reduction of iPS cells grown in keratinocyte media. We have performed spike-in experiments of undifferentiated GFP- labeled Oct4+H9 hES cells ranging up to 50% grown in co-culture with NHK in keratinocyte media for 14 days (Table 6). Cells were analyzed by FACS (detection sensitivity 1:100,000, see Table 6) with background levels approximately 0.62% (left column “0” Table 6). Background levels of Oct4+ GFP+ cells were detected when spiked until ES cells were added at 50:50. This showed a ˜100-fold reduction of pluripotent cells when grown in keratinocyte media for 2 weeks (iii) No detectable adherence of undifferentiated iPS cells on human dermis. GFP-tagged H9 cells do not incorporate into the DEBCT epithelial sheets in organotypic cultures even if they were present in as high as 50% of input cells. The homotypic desmosome adhesion molecules present on mature keratinocytes prevent H9 incorporation (detection sensitivity 1:100). These data provide rational limits for the percentage of remaining undifferentiated IPS cells in DEBCT. The fraction of undifferentiated cells after PI #4 selection (step M5) is approximately 0.3%. A two-week expansion in keratinocyte media (step M6) and then DEBCT graft assembly (step M7) reduces the number of iPS cells several logs resulting in too few iPS cells in a clinical dose (30 million iKCs) to cause tumor formation on a surface graft.

Lack of epidermal tumors in DEBCT 2014 Pilot Tumorigenicity in NSG xenografts. We have done preliminary testing for DEBCT tumorigenicity in the course of demonstrating DEBCT efficacy as we are grafting DEBCT on NSG immunocompromised mice. We have tested over 40 H9 ES and iPS cell-derived keratinocyte epithelial sheets for potency and tumorigenicity with an average duration of 1 month but up to 2 months in 20% of the grafts. Bread-loafing histological analysis indicates the grafts display normal epidermal characteristics and differentiation markers and do not display evidence of local invasion, neoplastic atypia, teratoma or squamous cell carcinoma formation. Moreover, we have shown using the same grafting system that human keratinocytes transformed by oncogenes into invasive SCCs will be visible within 4-6 weeks. These pilot tumorigenicity studies provide early safety assurances for keratinocyte manufacturing.

The Pilot Clinical Study to evaluate safety and tolerability of DEBCT is modeled after our previous studies using corrected keratinocyte sheets (LEAES), Trial NCT01263379, IND #13708, that was published previously.

Objectives and Planned Duration: This proof-of-concept, open-label study of one-time surgical application of DEBCT grafts for the treatment of eligible, chronic open wound sites in up to 5 RDEB patients. The primary endpoint is the safety and tolerability of DEBCT grafts for the treatment of chronic open wound sites in RDEB patients. Subjects will be followed for 12 months for safety. Subjects will continue to be followed at least annually for safety for 5 years in a separate long-term follow-up protocol. For safety monitoring, we will visually assess for SCC or neoplasms at the graft site.

Secondary endpoints include comparison of wound healing ≥50% from baseline at 3 months. Wound healing will be assessed and subsequently photographed and quantified using the Canfield system at 0, 3, 6 and 12 months. Exploratory endpoints include Investigator's Global Assessment (IGA) score (≥50%) of each graft/control site at 3 months, patient global impression scale of change at 3 months, and median of PROs assessing the subject's impression of itch and pain of each graft/control sites at 3 months. Assessment of biological function will include the expression of C7 and the presence of anchoring fibrils and longitudinal changes in PRO itch and pain scores.

The NC1 domain is generally accepted to be the most antigenic region on the C7 molecule. Therefore, an NC1[+] subject is less likely to develop immune reactions to sites of grafted autologous keratinocytes that express C7. RDEB subjects will be required to express the NC1 amino-terminal fragment of C7 (NC1[+], approximately 75-90% of patients), to be genotyped with confirmed recessive COL7A1 mutations, and to have no evidence of an immune response to C7 by indirect immunofluorescence (IIF).

iKCs sheets from subjects with RDEB will be grafted back to the original RDEB donor wounds. We intend to document type VII collagen protein correctly localized to the cutaneous basement membrane zone that is incorporated into ultrastructurally normal anchoring fibrils. We will monitor the durability of the type VII collagen by completing multiple skin biopsies during the subject's first year in the trial and possibly yearly after that period. We will monitor humoral or cellular immune responses for the subjects in order to provide information both about the likelihood of success of this approach as well as insight into any failure of durability. We plan to enroll subjects in a follow-up protocol for these assessments.

We will clinically examine the grafted areas to document the surface area of wound healing using Canfield software analysis and also investigator global assessment. We will examine the biological function of the iKC grafts by documenting production and duration of type VII collagen production. We will confirm the correct formation of anchoring fibrils by staining for type VII collagen using immunofluorescent analysis (IF) and immunoelectron microscopy (IEM). We will also examine the ultrastructural position of the anchoring fibrils using immunoelectron microscopy (IEM). We will monitor humoral immune responses for the RDEB recipients in order to provide information both about the likelihood of success of this approach as well as insight into any failure of durability. We will monitor subjects frequently in the first year and then most likely yearly for life for the development of neoplastic changes.

A specific subset of individuals with RDEB will be initially selected for this clinical trial. RDEB subjects will initially be required to be NC1[+], to have absent type VII collagen antibodies, to be genotyped with confirmed recessive COL7A1 mutations, and have no evidence of an immune response to type VII collagen.

There are several risks anticipated in this trial. Subjects could develop physical difficulties which could destroy individual grafts. These events include wound infections or physical trauma which removes the graft before it has attached. We have developed protocols for specific adverse events: immunologic rejection, systemic infection, and advancing epithelial surfaces.

Biopsies will be obtained from non- blistered skin for fibroblast culture, in order to manufacture the graft. The manufacturing aim is to produce and deliver up to six of the keratinocyte sheets for grafting (DEBCT). Approximately one to six of the 40-50 cm² epithelial sheets will be used in a single grafting session. The maximum total grafting surface area for all the graft sites will be 300 cm². At the screening visit we will select multiple potential wounds for grafting. We will follow these wounds clinically until the day of grafting/enrollment (Day 0). The decision on which sites to be grafted will not be finalized until Day 0. The determination that target wounds meet all graft criteria will be made at that time. The grafted wound areas will be selected by several criteria. The wounds should appear clean with adequate granulation tissue, adequate vascularization, and not appear infected. Bacterial cultures can be obtained from several wounds for culture and antibiotic sensitivities. Wound cultures may be repeated as needed based upon standard of care and medical judgment of the investigators and EB physician. The surface area should have a smooth texture that can accept a graft. The duration that the subject thinks that they have each wound will be recorded. The wounds will also need to meet mechanical requirements that decrease trauma to the grafted areas. Appropriate sites will be on the anterior and/or lateral trunk and/or upper and/or lower extremities in areas protected from frequent trauma or injury. Excluded areas will usually include the face, areas close to mucous membranes (genito-urinary, oral or anal mucosa), areas over joints and back. The distance from objective body landmarks will be identified and measured.

Grafting will be carried out under general anesthesia. Grafts will be labeled with the subject's name, medical record number (MRN), date of birth (DOB), and study number to confirm that the correct subject is receiving the correct grafts.

The grafting process will be: 1. All wounds will be gently cleansed with normal saline or povidone-iodine solution; 2. Overhanging epidermis, hyperkeratotic skin, or fibrinous material will be gently debrided with scalpel, scissors, or the timed surgery electrosurgical technique (or equivalent cauterization technique), or a combination of these at the grafting surgeon's discretion, in consultation with the EB physician; 3. iKC grafts will be applied to the wound beds and affixed with staples, suture, non-adhesive dressing (Mepitel, Adaptic®, Restore®, or other equivalent), and/or overlying dressing. A layer of topical antibiotics will be applied, with specific antibiotic determined by the grafting surgeon and EB physician; 4. During grafting, subjects will have a small (˜1 mm or less) tattoo dot placed at the corners of the grafts and edges of control areas. If the patient does not have useable body landmarks close to the wound site, the PI will choose a location for the surgeon to tattoo a small dot as a landmark.

### Example 3

### iKC Differentiation Protocol

Keratinocyte Differentiation Using Embryoid Bodies Protocol

### Materials

AGGREWELL™ 400 (24-well)
AGGREWELL™ Medium
AGGREWELL™ Rinsing Solution
Collagen I peptide 6-well plate
Reversible Cell Strainer, 37 μm
Cell strainer, 40 μm
Essential 6 (Gibco)
RevitaCell (ROCK Inhibitor, Gibco)
10 cm² tissue culture dishes
18 cir-1 coverslips (ThermoFisher)
12-well culture plates
Vitronectin (Gibco)
Retinoic Acid
BMP-4
Petri Dishes
Defined Keratinocyte Serum-Free Medium (Gibco)
Gentle Cell Dissociation Reagent (StemCell)
DMEM/F-12 (Gibco)

### Preparation.

Vitronectin (VTN-N, 100X) Coated Dishes and 12-well plates with 18 cir-1 coverslips. Coat dishes and 12-well plates with coverslips at least 1 hour before seeding embryoid bodies. Coated dishes and plates can be stored in the cold room wrapped in parafilm for up to 1 week.

AggreWell Medium+Revitacell or ROCK inhibitor-1-10 μM (+1 μM RA (AG+RV+RA) (100X, RV, ROCK Inhibitor). 24-well requires 2 mL per well, 6-well 5 mL per well.

AggreWell Medium+1 μM RA (AG+RA). 2 mL is required per well of a 6-well, prepare fresh.

Essential 6+1 μM RA+5 ng/uL BMP-4 (E6+RA+BMP4). 10 mL is required per coated dish, prepare fresh for every differentiation.

### Procedure

Embryoid Body Formation—Day-1. Harvest 80-90% confluent iPS cells that were grown in Stem Fit Basic02 attached to Laminin-511 using TrypLE Select 50/50 with PBS with an incubation time of 5-10 minutes at 37° C. to achieve single cell suspension. Pipette dissociated cells into a 50 mL falcon tube. Collect remaining cells by washing wells with PBS and then pipette into falcon tube, centrifuge for 5 min at 300 g. Aspirate supernatant, resuspend cells with 1 mL AG+RV+RA, count cells. Add 1.2×10⁶ cells to AggreWell. Add appropriate amount of AG+RV+RA to reach 2 mL in 24-well. Pipette up and down to ensure even distribution of cells. Centrifuge at 100 g for 3 minutes, check under microscope for homogenous distribution. Place in incubator at 37° C. for 20-24 hours.

Embryoid Body Harvest, Suspension Culture—Day 0. Not a required step) EBs can go directly to 10 cm plates for Differentiation. Check embryoid bodies under microscope to ensure proper formation. Using a P1000 pipette, GENTLY Pipette wells up and down and transfer EBs to reversible cell strainer. Wash wells with DMEM/F-12 three times with 1 mL and transfer to reversible cell strainer. Place new tube on cell strainer and FLIP. Pipette 5 mL of AG+RA through cell strainer to retrieve embryoid bodies. Using a P1000 Pipette transfer embryoid bodies to a low adherence 6-well plate with 2 mL of AG+RA (Alternatively to 10 cm non-adherent dish with 10 mL). Incubate for 48 hours.

Seed on to 10 cm2 vitronectin coated dishes and 12-well plates with coverslips, Culture with factors—Day 1-7. Use a 1 mL pipette to transfer embryoid bodies to 15 mL Falcon tube, allow them 5 mins to aggregate at the bottom. Prepare enough E6+RA+BMP4 media to feed cells 4 times, it must be made fresh for each differentiation (See Preparation). Aspirate media from tube, take great care to not aspirate the embryoid bodies. Resuspend embryoid bodies with 4 mL of E6+RA+BMP4. Evenly distribute 1 mL of a suspended embryoid bodies to 1 of 3 VTN-N coated 10 cm² dishes containing E6+RA+BMP4 using a 1 mL pipette. Add 11 mLs to the embryoid body suspension and distribute 1 mL of embryoid bodies to each well. After 7 days of E6+RA+BMP4, switch to Defined Keratinocyte Serum-Free Medium (DKSFM). Change DKSFM every other day for at least 45 days based on morphology.

Sorting and expansion of mature iPSC-derived Keratinocytes (iKC). Initiate dissociation of the 3-10 cm dishes by adding TrypLE for 5-10 minutes at 37° C. Harvest cells and pass through a 40 micron cell strainer. Count cells. Centrifuge at 300 g for 5 minutes. Aspirate supernatant, then resuspend up to 5×10⁶ cells with 100 uL of 3% GMP-grade FBS/PBS containing the conjugated CD49f (ITGA6) antibody (1:100 dilution). Add 1 uL of Propidium Iodide (PI) to detect cell viability. Sort positive control and negative control and set gates, keep gates set for subsequent sortings. Use P2 NHKs as internal controls for FACS and staining in CnT-Prime media. Sort into 50 mL conical tubes with DKSFM and P/S. Distribute 0.5×10⁶ cells into one well of a collagen I peptide coated 6-well plate (Optimal number of cells for initial seeding is tentative). Culture cells until they are 90% confluent, then passage with TrypLE with 50/50 PBS. Passage one well to three wells of a collagen I peptide coated 6-well plate. No more than two passages to get to dermis. After expansion, perform repeat cell composition assay using CD90, CD104, and TRA1-60. Repeat step 9 to end with 9 wells of iKCs for grafting.

### REFERENCES

Saeidian, A. H., et al. Seven novel COL7A1 mutations identified in patients with recessive dystrophic epidermolysis bullosa from Mexico. Clin Exp Dermatol 43, 579-584 (2018).

Siprashvili, Z., et al. Safety and Wound Outcomes Following Genetically Corrected Autologous Epidermal Grafts in Patients With Recessive Dystrophic Epidermolysis Bullosa. JAMA 316, 1808-1817 (2016).

Sebastiano, V., et al. Human COL7A1-corrected induced pluripotent stem cells for the treatment of recessive dystrophic epidermolysis bullosa. Sci Transl Med 6, 264ra163 (2014).

Umegaki-Arao, N., et al. Induced pluripotent stem cells from human revertant keratinocytes for the treatment of epidermolysis bullosa. Sci Transl Med 6, 264ra164 (2014).

Kogut, I., Roop, D. R. & Bilousova, G. Differentiation of human induced pluripotent stem cells into a keratinocyte lineage. Methods Mol Biol 1195, 1-12 (2014).

Siprashvili, Z., et al. Long-term type VII collagen restoration to human epidermolysis bullosa skin tissue. Hum Gene Ther 21, 1299-1310 (2010).

Karagiannis, P., et al. Induced Pluripotent Stem Cells and Their Use in Human Models of Disease and Development. Physiol Rev 99, 79-114 (2019).

Hirai, H., et al. Radical acceleration of nuclear reprogramming by chromatin remodeling with the transactivation domain of MyoD. Stem Cells 29, 1349-1361 (2011).

Mali, P., Esvelt, K. M. & Church, G. M. Cas9 as a versatile tool for engineering biology. Nature methods 10, 957-963 (2013).

Mali, P., et al. RNA-guided human genome engineering via Cas9. Science 339, 823-826 (2013).

Jinek, M., et al. RNA-programmed genome editing in human cells. Elife 2, e00471 (2013).

Vakulskas, C. A., et al. A high-fidelity Cas9 mutant delivered as a ribonucleoprotein complex enables efficient gene editing in human hematopoietic stem and progenitor cells. Nat Med 24, 1216-1224 (2018).

Nakagawa, M., et al. A novel efficient feeder-free culture system for the derivation of human induced pluripotent stem cells. Sci Rep 4, 3594 (2014).

Pattison, J. M., et al. Retinoic acid and BMP4 cooperate with p63 to alter chromatin dynamics during surface epithelial commitment. Nat Genet 50, 1658-1665 (2018).

Lorenz, K., Rupf, T., Salvetter, J. & Bader, A. Enrichment of human beta 1 bri/alpha 6 bri/CD71 dim keratinocytes after culture in defined media. Cells Tissues Organs 189, 382-390 (2009).

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements, and variations are considered to be within the scope of this disclosure. The materials, methods, and examples provided here are representative of particular embodiments, are exemplary, and are not intended as limitations on the scope of the disclosure.

The scope of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A one-step method of generating genetically corrected, induced pluripotent cells for regenerative therapy, the method comprising:
obtaining a population of somatic cells from an individual having a mutation at a target locus of interest for genetic correction;
contacting the population of somatic cells with an effective dose of preformed ribonuclear protein (RNPs) complexes comprising a guide RNA specific for the locus of interest and CRISPR/cas9; and a repair sequence template specific for the locus of interest to generate genetically corrected cells;
maintaining the population of genetically corrected cells in feeder-free, xeno-free medium for a period of from 2 to 6 days;
contacting the population of cells with modified mRNA encoding reprogramming factors to induce the cells to pluripotency;
maintaining the population of cells in feeder-free, xeno-free medium to generate colonies of iPSC.

2. The method of claim 1, wherein the reprogramming factors comprise synthetic capped mRNAs containing modified nucleobases for M3O, Sox2, Klf4, cMyc, and Lin28A.

3. The method of claim 1 or claim 2, wherein the somatic cells are fibroblasts.

4. The method of any of claims 1-3, wherein the population of somatic cells is autologous relative to an individual selected for treatment.

5. The method of any of claims 1-4, wherein the iPSC are expanded in culture.

6. The method of any of claims 1-5, wherein the iPSC are frozen prior to expansion.

7. The method of any of claims 1-6, wherein the iPSC are differentiated to a somatic cell type.

8. The method of claim 7, wherein the iPSC are differentiated to iKCs.

9. The method of claim 8, wherein the iKCs are selected for expression of CD49f.

10. The method of claim 9, wherein the CD49f+ iKCs are manufactured as a sheet for engraftment.

11. The method of any of claims 1-10, wherein the somatic cells are obtained from an individual with Epidermolysis Bullosa (EB).

12. The method of claim 11, wherein the locus of interest for genetic correction is COL7A1.

13. The method of any of claims 1-10, wherein the somatic cells are obtained from an individual with a genodermatosis.

14. The method of claim 13, wherein the genodermatosis is Ectodermal Dysplasia.

15. The method of claim 13, wherein the genodermatosis is Hypohidrotic ectodermal dysplasia.

16. The method of claim 13, wherein the genodermatosis is Hidrotic ectodermal dysplasia.

17. The method of claim 13, wherein the genodermatosis is White sponge nevus.

18. The method of claim 13, wherein the genodermatosis is Hereditary, benign, intraepithelial-dyskeratosis.

19. The method of claim 13, wherein the genodermatosis is Pachyonychia congenita.

20. The method of claim 13, wherein the genodermatosis is Dyskeratosis congenita.

21. The method of claim 13, wherein the genodermatosis is Xeroderma pigmentosum.

22. The method of claim 13, wherein the genodermatosis is Incontinentia pigmenti.

23. The method of claim 13, wherein the genodermatosis is Keratosis follicularis.

24. The method of claim 13, wherein the genodermatosis is Warty dyskeratoma.

25. The method of claim 13, wherein the genodermatosis is Peutz-Jeghers syndrome.

26. The method of claim 13, wherein the genodermatosis is Ehlers-Danlos syndrome.

27. The method of claim 13, wherein the genodermatosis is Tuberous sclerosis.

28. The method of claim 13, wherein the genodermatosis is Netherton syndrome.

29. A method of treating a genodermatosis in a subject, the method comprising
obtaining from the subject a population of somatic cells;
contacting the population of somatic cells with an effective dose of preformed ribonuclear protein (RNPs) complexes comprising a guide RNA specific for a causative disease gene and CRISPR/cas9; and a repair sequence template specific for the causative disease gene to generate genetically corrected cells;
maintaining the population of genetically corrected cells in feeder-free, xeno-free medium for a period of from 2 to 6 days;
contacting the population of cells with modified mRNA encoding reprogramming factors to induce the cells to pluripotency;
differentiating the pluripotent cells to iKCs.
culturing the iKCs to form a keratinocyte sheet; and
transplanting a graft of the iKC sheet to a skin wound bed of the subject.

30. The method of claim 29, wherein the genodermatosis is Epidermolysis bullosa.

31. The method of claim 29, wherein the genodermatosis is Ectodermal Dysplasia.

32. The method of claim 29, wherein the genodermatosis is Hypohidrotic ectodermal dysplasia.

33. The method of claim 29, wherein the genodermatosis is Hidrotic ectodermal dysplasia.

34. The method of claim 29, wherein the genodermatosis is White sponge nevus.

35. The method of claim 29, wherein the genodermatosis is Hereditary, benign, intraepithelial-dyskeratosis.

36. The method of claim 29, wherein the genodermatosis is Pachyonychia congenita.

37. The method of claim 29, wherein the genodermatosis is Dyskeratosis congenita.

38. The method of claim 29, wherein the genodermatosis is Xeroderma pigmentosum.

39. The method of claim 29, wherein the genodermatosis is Incontinentia pigmenti.

40. The method of claim 29, wherein the genodermatosis is Keratosis follicularis.

41. The method of claim 29, wherein the genodermatosis is Warty dyskeratoma.

42. The method of claim 29, wherein the genodermatosis is Peutz-Jeghers syndrome.

43. The method of claim 29, wherein the genodermatosis is Ehlers-Danlos syndrome.

44. The method of claim 29, wherein the genodermatosis is Tuberous sclerosis.

45. The method of claim 29, wherein the genodermatosis is Netherton syndrome.

46. The method of claim 29, wherein the reprogramming factors comprise synthetic capped mRNAs containing modified nucleobases for M3O, Sox2, Klf4, cMyc, and Lin28A.

47. The method of any of claims 29-46, wherein the iPSC are expanded in culture.

48. The method of any of claims 29-47, wherein the iPSC are frozen prior to differentiation to iKCs.

49. The method of any of claims 29-48, wherein the iKCs are selected for expression of CD49f prior to formation of the keratinocyte sheet.

50. The method of any one of claims 29-49, wherein the wound is free of non-corrected wound bed keratinocytes.

51. The method of any one of claims 29-50, wherein the wound is treated to ablate the non-corrected wound bed keratinocytes.

52. The method or the use of any one of claim 29 or 46-51, wherein the subject suffers from Recessive Dystrophic Epidermolysis Bullosa (RDEB).

53. A pharmaceutical formulation comprising a cell population produced the method of any of claims 1-28.
